(19) United States
(12) Patent Application Publication
(10) Pub. No.: US 2024/0245632 A1
(43) Pub. Date: Jul. 25, 2024

(54) METHODS OF TREATING SUBSTANCE USE DISORDERS USING MESCALINE

(71) Applicant: Journey Colab Corp., San Francisco, CA (US)

(72) Inventors: Jeeshan Chowdhury, San Francisco, CA (US); Derek Londesbrough, Hartlepool (GB); Christopher Gill, Sedgefield (GB)

(21) Appl. No.: 18/564,553

(22) PCT Filed: May 27, 2022
(86) PCT No.: PCT/US22/31423

§ 371 Date: Nov. 27, 2023

Related U.S. Application Data
(60) Provisional application No. 63/345,602, filed on May 25, 2022.
(60) Provisional application No. 63/314,129, filed on Feb. 25, 2022.
(60) Provisional application No. 63/270,989, filed on Oct. 22, 2021.
(60) Provisional application No. 63/194,863, filed on May 28, 2021.

(51) Int. Cl. A61K 31/137; A61K 9/00

(52) U.S. Cl. CPC A61K 31/137; A61K 9/0053

(57) ABSTRACT

The present disclosure provides methods of treating substance use disorders comprising the oral administration mescaline or a salt thereof. In another aspect, the oral administration comprises a pharmaceutical composition comprising synthetic mescaline. The synthetic mescaline, mescaline or salts thereof, and pharmaceutical compositions disclosed herein, provide several advantages over plant extracted mescaline. For example, plant extractions can have varying concentrations of active mescaline leading to dosing variability and potency. In additional embodiments, the administration is accompanied with a preparatory therapy session. In another aspect, the method further comprises a plurality of treatment steps to aid in the efficacy of treating substance use disorders.

### CROSS-REFERENCE

This application claims the benefit of U.S. Provisional Patent Application No. 63/194,863 filed on May 28, 2021, U.S. Provisional Patent Application No. 63/270,989 filed on Oct. 22, 2021, U.S. Provisional Patent Application No. 63/314,129 filed on Feb. 25, 2022, and U.S. Provisional Patent Application No. 63/345,602 filed on May 25, 2022, the entire contents of each of which are incorporated herein by reference.

### BACKGROUND OF THE INVENTION

Substance use disorders (SUDs) effect over 20 million adults and over 440,000 adolescents in the United States. Annually, it is estimated that 70,000 Americans die from drug overdoses, and that SUDs cost American society more than $750 billion annually in lost workplace productivity, healthcare expenses, and crime related costs. Drug-related deaths in the United States have more than tripled since 2000, and there are more deaths, illness, and disabilities from substance use than from any other preventable health condition, to the extent that one in four deaths in the United States is currently attributable to alcohol, tobacco, and illicit or prescription drug use. To date there are only three FDA approved drugs for the treatment of alcohol use disorder—disulfiram, naltrexone, and acamprosate—and only three approved drugs for the treatment of Opioid Use Disorder—buprenorphine, methadone, and naltrexone—and none for many other SUDs, highlighting the need for new and effective treatments for SUDs.

### SUMMARY OF THE INVENTION

In certain aspects, the present disclosure provides a method for treating a substance use disorder, comprising (a) identifying a subject who has a substance use disorder; and (b) orally administering a pharmaceutical composition comprising synthetic mescaline or a salt thereof to the subject and accompanying said administration with a therapy session. In some embodiments, step (b) is monitored by a licensed healthcare professional. In some embodiments, the synthetic mescaline was produced in accordance with GMP guidelines. In some embodiments, the synthetic mescaline was produced in a GMP facility. In some embodiments, the synthetic mescaline comprises less than 5% impurities. In some embodiments, the synthetic mescaline comprises less than 1% impurities. In some embodiments, the impurities are selected from plant cells, plant tissue, and an alkaloid. In some embodiments, the alkaloid is a naturally occurring alkaloid. In some embodiments, the synthetic mescaline is represented by Formula (IV):

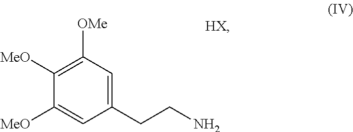 [SMILES: COc1cc(CCN)cc(OC)c1OC]

wherein HX is an acid and X represents the conjugate base of the acid. In some embodiments, the pharmaceutical composition comprises an acid addition salt of mescaline selected from an HCl addition salt and a sulfuric acid addition salt. In some embodiments, the pharmaceutical composition comprises mescaline HCl. In some embodiments, the synthetic mescaline is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the synthetic mescaline is characterized by two or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the synthetic mescaline is characterized by three or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the pharmaceutical composition comprises from 50-2000 mg of synthetic mescaline or a salt thereof. In some embodiments, the pharmaceutical composition comprises from 50-700 mg of synthetic mescaline or a salt thereof. In some embodiments, the pharmaceutical composition is in a tablet or a capsule. In some embodiments, 80% or more of the pharmaceutical composition comprises the synthetic mescaline or salt thereof. In some embodiments, the method further comprises a preparatory therapy session prior to step (b) to prepare the subject for the possible psychological and physiological effects of the administering of step (b). In some embodiments, the method further comprises obtaining informed written consent from the subject prior to step (b). In some embodiments, the subject is instructed to refrain from eating food for at least 10 hours prior to the administration of step (b). In some embodiments, step (b) further comprises physiological monitoring of the subject. In some embodiments, the physiological monitoring of the subject comprises monitoring of a physiological parameter selected from supine blood pressure, pulse rate, respiratory rate, and body temperature. In some embodiments, the physiological monitoring of the subject comprises monitoring of a plasma biomarker selected from brain-derived neurotrophic factor (BDNF) and hypothalamic-pituitary-adrenal (HPA)-axis endocrine measures. In some embodiments, step (b) further comprises psychological monitoring of the subject. In some embodiments, the psychological monitoring of the subject comprises monitoring for negative psychological reactions to treatment. In some embodiments, administering said treatment attenuates substance intake by said subject by about 20% or more as compared with the amount of substance intake before said treatment. In some embodiments, for step (a), said subject has reduced substance consumption for at least 1, 2, 3, 4, 5, 6, or 7 days prior to step (b). In some embodiments, the subject has reduced substance consumption for 5-12 days. In some embodiments, the method further comprises a mescaline treatment holiday of from 2 weeks to 12 months. In some embodiments, the therapy session comprises a practitioner and a plurality of subjects, wherein the practitioner abstains from the oral administration of the pharmaceutical composition. In some embodiments, the therapy session comprises the practitioner and at least one subject. In some embodiments, the therapy session comprises psychosocial therapy. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is an alcohol use disorder selected from alcohol abuse, alcohol dependence, and alcoholism.

In another aspect, the present disclosure provides a method of for treating a substance use disorder, comprising administering to a subject in need thereof an oral formulation comprising a mescaline salt, wherein the oral formulation comprises from 50-2000 mg of the mescaline salt and following said administering with a mescaline treatment holiday of 2 weeks to 12 months. In some embodiments, the method further comprises a therapy session after the administration of the oral formulation. In some embodiments, the therapy session comprises a practitioner and a plurality of subjects, wherein the practitioner abstains from the oral administration of the oral formulation. In some embodiments, the therapy session comprises the practitioner and at least one subject. In some embodiments, the therapy session comprises psychosocial therapy. In some embodiments, administering said treatment attenuates substance intake by said subject by about 20% or more as compared with the amount of substance intake before said treatment. In some embodiments, said subject has reduced substance consumption for at least 1, 2, 3, 4, 5, 6, or 7 days prior to treatment. In some embodiments, the subject has reduced substance consumption for 5-12 days prior to treatment. In some embodiments, the mescaline salt is selected from the HCl addition salt and sulfuric acid addition salt. In some embodiments, the mescaline salt is mescaline HCl. In some embodiments, the oral formulation comprises less than 5% impurities. In some embodiments, the impurity is selected from plant cells, plant tissue, and naturally occurring alkaloids. In some embodiments, the alkaloid is a naturally occurring alkaloid. In some embodiments, the oral formulation is in a tablet or a capsule. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is an alcohol use disorder selected from alcohol abuse, alcohol dependence, and alcoholism.

In another aspect, the present disclosure provides a method for treating a substance use disorder comprising, orally administering a pharmaceutical composition comprising a mescaline drug substance and a pharmaceutically acceptable excipient to the subject and accompanying said administration with a therapy session, wherein the pharmaceutical composition comprises less than 5% impurities. In some embodiments, the mescaline drug substance has less than 5% impurities. In some embodiments, the pharmaceutical composition comprises less than 5% impurities. In some embodiments, the impurity is selected from plant cells, plant tissue, and an alkaloid. In some embodiments, the alkaloid is a naturally occurring alkaloid. In some embodiments, the method further comprises a mescaline treatment holiday of from 2 weeks to 12 months. In some embodiments, the therapy session comprises a practitioner and a plurality of subjects, wherein the practitioner abstains from the oral administration of the oral formulation. In some embodiments, the therapy session comprises the practitioner and at least one subject. In some embodiments, the therapy session comprises psychosocial therapy. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is an alcohol use disorder selected from alcohol abuse, alcohol dependence, and alcoholism.

In another aspect, the present disclosure provides a method of treating a substance use disorder comprising: (a) administering a first treatment comprising mescaline or a salt thereof and accompanying said first treatment with a first therapy session; (b) administering a second treatment 5-12 days after said first treatment, said second treatment comprising mescaline or a salt thereof and accompanying said second treatment with a second therapy session. In some embodiments, the method further comprises one or more maintenance treatments comprising mescaline or a salt thereof and accompanying said maintenance treatment with a maintenance therapy session. In some embodiments, the first therapy session, the second therapy session, and the maintenance therapy session comprises a practitioner and a plurality of subjects, wherein the practitioner abstains from the administration comprising mescaline or a salt thereof. In some embodiments, the first therapy session, the second therapy session, and the maintenance therapy session comprises the practitioner and at least one subject. In some embodiments, the first therapy session, the second therapy session, and the maintenance therapy session comprises psychosocial therapy. In some embodiments, the mescaline or a salt thereof is administered as a pharmaceutical composition comprising mescaline or a salt thereof and a pharmaceutically acceptable excipient, wherein the pharmaceutical composition comprises less than 5% impurities. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is an alcohol use disorder selected from alcohol abuse, alcohol dependence, and alcoholism.

In another aspect, the present disclosure provides a method of treating a substance use disorder, comprising: (a) identifying a subject who has a substance use disorder; and (b) orally administering a pharmaceutical composition comprising mescaline or a salt thereof to the subject and accompanying said administration with a therapy session. In some embodiments, administering said treatment attenuates substance intake by said subject by about 20% or more as compared with the amount of substance intake before said treatment. In some embodiments, for step (a), said subject has reduced substance consumption for at least 1, 2, 3, 4, 5, 6, or 7 days prior to step (b). In some embodiments, the subject has reduced substance consumption for 5-12 days. In some embodiments, the method further comprises a mescaline treatment holiday of from 2 weeks to 12 months. In some embodiments, the pharmaceutical composition comprises an acid addition salt of mescaline selected from the HCl addition salt and sulfuric acid addition salt. In some embodiments, the pharmaceutical composition comprises mescaline HCl. In some embodiments, the pharmaceutical composition comprises from 50-800 mg of mescaline or a salt thereof. In some embodiments, the therapy session comprises a practitioner and a plurality of subjects, wherein the practitioner abstains from the oral administration of the pharmaceutical composition comprising mescaline or salt thereof. In some embodiments, the therapy session comprises the practitioner and at least one subject. In some embodiments, the therapy session comprises psychosocial therapy. In some embodiments, the pharmaceutical composition comprises less than 5% impurities. In some embodiments, the impurity is selected from plant cells, plant tissue, and an alkaloid. In some embodiments, the alkaloid is a naturally occurring alkaloid. In some embodiments, the pharmaceutical composition is in a tablet or a capsule. In some embodiments, 80% or more of the pharmaceutical composition comprises mescaline or salt thereof. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is an alcohol use disorder selected from alcohol abuse, alcohol dependence, and alcoholism.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “substance use disorder” refers to substance related disorders as defined by the Diagnostic and Statistical Manual of Mental Disorders (DSM-5). Criteria for evaluating substance use disorders are as defined by DSM-5.

The term “substance” refers to a drug substance as described herein. In some embodiments, a substance is selected from: alcohol, cannabis, caffeine, phencyclidine, inhalant, opioid, sedative, hypnotic, anxiolytic, stimulant and tobacco. In some embodiments, the substance as disclosed herein is defined by DSM-5.

The term “substance related disorder” refers to two groups of disorders: substance use disorder and substance-induced disorders as defined by DSM-5. Substance use disorders, as used herein, refer to alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, or tobacco use disorder. In some embodiments, the substance use disorder is a substance-induced disorder.

The term “alcohol use disorder” or “alcohol-related disorders” may refer to a diagnosis of disorders as defined under substance-related disorders and addictive disorders by DSM-5.

A clinician or medical professional may diagnose the severity of a substance use disorder using criteria or symptoms as defined by DSM-5. A “mild” diagnosis refers to two or three, symptoms or criteria as defined by DSM-5. A “moderate” diagnosis refers to four or five, symptoms or criteria as defined by DSM-5. A “severe” diagnosis refers to six or more, symptoms or criteria as defined by DSM-5.

The term “alcohol consumption” are defined using standards as defined by the National Institute on Alcohol Abuse and Alcoholism (NIAAA) or risk drinking levels as defined by the World Health Organization (WHO). In certain embodiments, a practitioner may evaluate a subject's reduction in alcohol consumption.

The term “heavy drinking days” are days when a male subject consumes more than four standard drinks per day, or a female subject consumes more than three “standard drinks” per day. A “standard drink” is defined as 1 oz. of liquor, 12 oz. of beer, or 5 oz. of wine. In certain embodiments, alcohol consumption is measured using heavy drinking days. In certain embodiments, a reduction in alcohol consumption is a reduction in the number of heavy drinking days.

The term “risk drinking levels” are defined as either “very high risk level,” “high risk level,” “moderate risk level,” or “low risk level.” In some embodiments, the reduction in alcohol consumption is a reduction in the risk drinking level. In some embodiments, the reduction in alcohol consumption is a reduction within the risk drinking level. In certain embodiments, the reduction in alcohol consumption is measured using the risk drinking level.

The term “very high risk level” is defined as greater than 100 g of alcohol for a male subject and greater than 60 g of alcohol for a female subject. In some embodiments, “very high risk level” is defined as greater than 7.1 standard drinks for a male subject and greater than 4.3 standard drinks for a female subject.

The term “high risk level” is defined as 60-100 g of alcohol for a male subject and 40-60 g of alcohol for a female subject. In some embodiments, “high risk level” is defined as 4.3-7.1 standard drinks for a male subject 2.9-4.3 standard drinks for a female subject.

The term “moderate risk level” is defined as 40-60 g of alcohol for a male subject and 20-40 g of alcohol for a female subject. In some embodiments, “moderate risk level” is defined as 2.9-4.3 standard drinks for a male subject and 1.4-2.9 standard drinks for a female subject.

The term “low risk level” is defined as 1-40 g of alcohol for a male subject and 1-20 g of alcohol for a female subject. In some embodiments, “low risk level” is defined as less than 2.9 standard drinks for a male subject and less than 1.4 standard drinks for a female subject.

The term “binge drinking” are defined as standards provided by the National Institute on Alcohol Abuse and Alcoholism (NIAAA) or as defined by the World Health Organization (WHO). In some embodiments, binge drinking is defined as using the standards provided by the National Institute on Alcohol Abuse and Alcoholism (NIAAA). In some embodiments, “binge drinking” is defined as a pattern of drinking alcohol that brings blood alcohol concentration (BAC) to 0.08 percent or higher. In some embodiments, binge drinking is defined as a pattern of drinking alcohol that brings blood alcohol concentration (BAC) to 0.08 grams of alcohol per deciliter or higher. For a typical adult, this pattern corresponds to consuming 5 or more drinks (male), or 4 or more drinks (female), in about 2 hours. In certain embodiments, a reduction in the pattern of drinking is a reduction in binge drinking. In some embodiments, binge drinking is defined as using the standards proved by the World Health Organization (WHO). In some embodiments, binge drinking is defined as heavy episodic drinking adults (aged ≥15 years) who consume at least 60 grams or more of pure alcohol at least once a week. In certain embodiments, a reduction in the consumption of pure alcohol once a week is a reduction in binge drinking.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the disclosure or a prodrug of a compound of the disclosure to the individual in need.

The term “plant tissue” refers to a part, or the whole, of a plant that had been removed.

The term “plant cells” means a more than one cell from an organism belonging to the Plante kingdom.

The efficacy of mescaline or salt thereof, oral formulation, or pharmaceutical compositions, as disclosed herein, may be measured as a score. The scores correspond to peak psychedelic experience, mystical experience, and the like. The occurrence of a peak psychedelic experience can be identified through achievement of at least 60% of the maximum possible score in each of the four subscales (mystical, positive mood, transcendence of time and space, and ineffability) of the 30-item revised Mystical Experience Questionnaire (MEQ30) as described in (Barrett F. S., J Psychopharmacol., 2015, 29, 1182-90, the entirety of which is incorporated by reference) or is identified through achievement of at least 60% of the maximum possible score of the Oceanic Boundlessness (OBN) dimension of the Altered States of Consciousness (ASC) questionnaire (R. L. et al., Front Pharmacol., 2018, 8, 974, the entirety of which is incorporated by reference) or through achievement of a Peak Psychedelic Experience Questionnaire (PPEQ) Total Score of at least 75.

The efficacy of treatment, as disclosed herein, may be assessed using Food and Drug Administration (FDA) draft guidance as provided under “Alcoholism: Developing Drugs for Treatment” (February 2015).

The term “effective amount” or “therapeutically effective amount” refers to that amount of a compound or salt described herein that is sufficient to affect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

The term “practitioner,” “medical practitioner,” “clinician,” “licensed healthcare professional” or “medical professional” is are defined as trained individuals in the medicinal arts and the like. For example, trained individuals can include, but are not limited to medical doctors, psychiatrists, clinical or counseling psychologists, nurse practitioners, physician assistants, registered nurses, clinical scientists, addiction specialists, substance abuse counselors, or therapists.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about,” as used herein, includes the recited number ±10%. Thus, “about 10” means 9 to 11.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “at most”, “less than,” or “less than or equal to” precedes the first numerical value in a series of two or more numerical values, the term “no more than,” “at most”, “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “decreased” or “decrease,” as used herein, generally mean a decrease by a statistically significant amount. In some embodiments, “decreased” or “decrease” means a reduction by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease from 10% to 100% as compared to a reference level. In the context of a marker or symptom, by these terms is meant a statistically significant decrease in such level. The decrease can be, for example, at least 10%, at least 20%, at least 30%, at least 40% or more.

The section headings used herein are for organizational purposes only and are not to be construed as limiting the subject matter described.

Any aspect or embodiments described herein can be combined with any other aspect or embodiments as disclosed herein.

### (a) Methods of Treating Substance Use Disorders

### Treating Substance Use Disorders Based on Reduced Substance Consumption

In certain aspects the present disclosure provides methods of treating substance use disorders. In some embodiments, the method for treating a substance use disorder comprises (a) identifying a subject who has a substance use disorder; and (b) orally administering a pharmaceutical composition comprising mescaline or a salt thereof to the subject and accompanying said administration with a therapy session.

In some embodiments, the subject is identified with a substance use disorder by a medical professional using symptoms or criteria as defined by DSM-5. In some embodiments, the substance use disorder in a subject is diagnosed by a medical professional. In some embodiments, the substance use disorder in a subject is diagnosed as a mild substance use disorder. In some embodiments, the substance use disorder in a subject is diagnosed as a moderate substance use disorder. In some embodiments, the substance use disorder in a subject is diagnosed as a severe substance use disorders.

In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalant use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism.

In some embodiments, administering said treatment attenuates substance intake by said subject by about 20% or more as compared with the amount of substance intake before said treatment. In some embodiments, the administering said treatment attenuates substance intake by said subject, by about 30%, about 40%, about 50%, about 60%, about 70%, about 80%, about 90%, or about 95% or more as compared with the amount of substance intake before said treatment. In some embodiments, the administering said treatment attenuates substance intake by said subject, by at least 20%, at least 30%, at least 40%, at least 50%, at least 60%, at least 70%, at least 80%, at least 90%, or at least 95% as compared with the amount of substance intake before said treatment.

In some embodiments, for step (a), said subject has reduced substance consumption for at least 1, 2, 3, 4, 5, 6, or 7 days prior to step (b). In some embodiments, for step (a), said subject has reduced substance consumption for at least 3, 4, 5, 6, or 7 days prior to step (b). In some embodiments, for step (a), said subject has reduced substance consumption for more than 1, 2, 3, 4, 5, 6, or 7 days prior to step (b). In some embodiments, for step (a), said subject has reduced substance consumption for more than 3, 4, 5, 6, or 7 days prior to step (b). In some embodiments, for step (a), said subject has reduced substance consumption for at least 2 days prior to step (b). In some embodiments, for step (a), said subject has reduced substance consumption for at least 3 days prior to step (b). In some embodiments, for step (a), said subject has reduced substance consumption for at least 4 days prior to step (b). In some embodiments, for step (a), said subject has reduced substance consumption for at least 5 days prior to step (b). In some embodiments, for step (a), said subject has reduced substance consumption for at least 6 days prior to step (b). In some embodiments, for step (a), said subject has reduced substance consumption for at least 7 days prior to step (b). In some embodiments, for step (a), said subject has reduced substance consumption for more than 2 days prior to step (b). In some embodiments, for step (a), said subject has reduced substance consumption for more than 3 days prior to step (b). In some embodiments, for step (a), said subject has reduced substance consumption for more than 4 days prior to step (b). In some embodiments, for step (a), said subject has reduced substance consumption for more than 5 days prior to step (b). In some embodiments, for step (a), said subject has reduced substance consumption for more than 6 days prior to step (b). In some embodiments, for step (a), said subject has reduced substance consumption for more than 7 days prior to step (b).

In some embodiments, the subject has reduced substance consumption for 1-12 days. In some embodiments, the subject has reduced substance consumption for 2-12 days. In some embodiments, the subject has reduced substance consumption for 3-12 days. In some embodiments, the subject has reduced substance consumption for 4-12 days. In some embodiments, the subject has reduced substance consumption for 5-12 days. In some embodiments, the subject has reduced substance consumption for 5-11 days. In some embodiments, the subject has reduced substance consumption for 5-10 days. In some embodiments, the subject has reduced substance consumption for 5-9 days. In some embodiments, the subject has reduced substance consumption for 5-8 days. In some embodiments, the subject has reduced substance consumption for 5-7 days. In some embodiments, the subject has reduced substance consumption for 5-6 days. In some embodiments, the subject has reduced substance consumption for 6-12 days. In some embodiments, the subject has reduced substance consumption for 7-12 days. In some embodiments, the subject has reduced substance consumption for 8-12 days. In some embodiments, the subject has reduced substance consumption for 9-12 days. In some embodiments, the subject has reduced substance consumption for 10-12 days. In some embodiments, the subject has reduced substance consumption for 11-12 days.

In some embodiments, further comprising a mescaline treatment holiday of from 2 weeks to 12 months. In some embodiments, the mescaline treatment holiday is from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 2 weeks to 2 months. In some embodiments, the mescaline treatment holiday is from 2 weeks to 1 months. In some embodiments, the mescaline treatment holiday is from 2 weeks to 4 weeks. In some embodiments, the mescaline treatment holiday is from 2 weeks to 3 weeks. In some embodiments, the mescaline treatment holiday is from 1 month, 2 months, 3 months, 4, month, 5 months, 6 months, 7 month, 8 months, 9 months, 10 months, 11 months, or 12 months. In some embodiments, the mescaline treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the pharmaceutical composition comprises an acid addition salt of mescaline selected from the HCl addition salt and sulfuric acid addition salt. In some embodiments, the pharmaceutical composition comprises mescaline HCl.

In some embodiments, the pharmaceutical composition comprises from 50-700 mg of mescaline or salt thereof. In some embodiments, the pharmaceutical composition comprises from about 50-600 mg of mescaline or salt thereof. In some embodiments, the pharmaceutical composition comprises from about 50-500 mg of mescaline or salt thereof. In some embodiments, the pharmaceutical composition comprises from about 50-400 mg of mescaline or salt thereof. In some embodiments, the pharmaceutical composition comprises from about 50-300 mg of mescaline or salt thereof. In some embodiments, the pharmaceutical composition comprises from about 50-200 mg of mescaline or salt thereof. In some embodiments, the pharmaceutical composition comprises from about 50-700 mg of mescaline or salt thereof. In some embodiments, the pharmaceutical composition comprises from about 50-700 mg of mescaline or salt thereof. In some embodiments, the pharmaceutical composition comprises from about 50-700 mg of mescaline or salt thereof. In some embodiments, the pharmaceutical composition comprises from about 50-700 mg of mescaline or salt thereof. In some embodiments, the pharmaceutical composition comprises from about 50-700 mg of mescaline or salt thereof. In some embodiments, the pharmaceutical composition comprises about 50 mg, about 100 mg, about 200 mg, about 300 mg, about 400 mg, about 500 mg, about 600 mg, or about 700 mg of mescaline or a salt thereof. In some embodiments, the pharmaceutical composition comprises at least 50 mg, at least 100 mg, at least 200 mg, at least 300 mg, at least 400 mg, at least 500 mg, at least 600 mg, or at least 700 mg of mescaline or a salt thereof. In some embodiments, the pharmaceutical composition comprises at most 50 mg, at most 100 mg, at most 200 mg, at most 300 mg, at most 400 mg, at most 500 mg, at most 600 mg, or at most 700 mg of mescaline or a salt thereof.

In some embodiments, the therapy session comprises a practitioner and a plurality of subjects, wherein the practitioner abstains from the oral administration of the pharmaceutical composition comprising mescaline or a salt thereof. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialists, a substance abuse counselor, a therapist, or any combination thereof. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities. In some embodiments, the impurity is selected from plant cells, plant tissue, and naturally occurring alkaloids.

In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by:

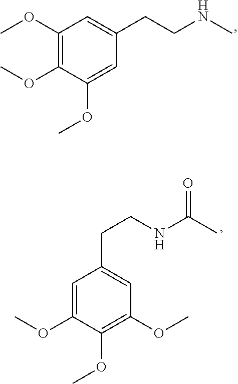

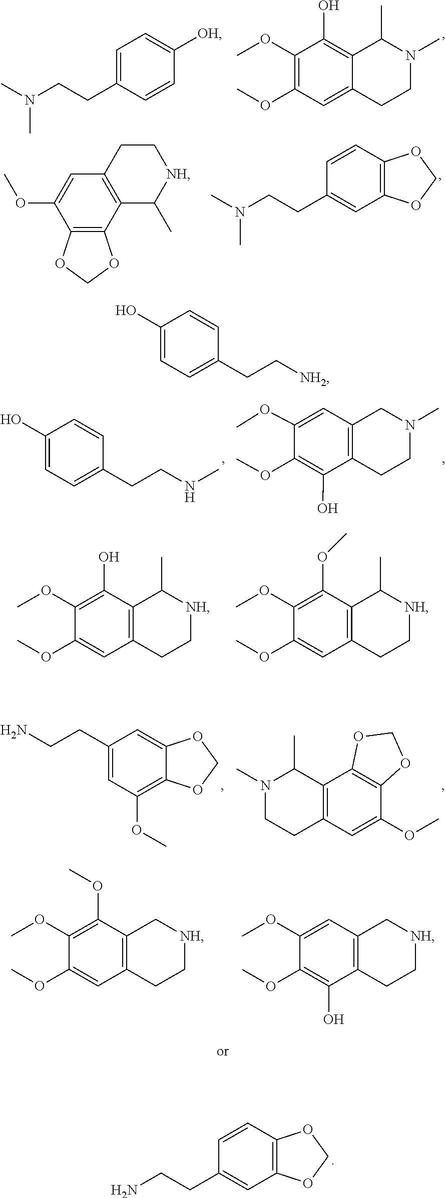

In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form. In some embodiments, the pharmaceutical composition in the tablet comprises less than 5% impurities as disclosed herein. In some embodiments, the pharmaceutical composition in the capsule comprises less than 5% impurities as disclosed herein.

In some embodiments, 80% or more of the pharmaceutical composition comprises mescaline or salt thereof. In some embodiments, 90% or more of the pharmaceutical composition comprises mescaline or salt thereof. In some embodiments, 95% or more of the pharmaceutical composition comprises mescaline or salt thereof. In some embodiments, 80% or more, 85% or more, 90% or more, 95% or more, or 99% or more of the pharmaceutical composition comprises mescaline or salt thereof. In some embodiments, at least 80%, at least 85%, at least 90%, at least 95%, or at least 99% of the pharmaceutical composition comprises mescaline or salt thereof.

### Treating Substance Use Disorders Based on Mescaline Treatment Holiday

In some aspects the present disclosure provides methods of treating substance use disorders based on a designated mescaline drug holiday. In another embodiments, the method of treating a substance use disorder comprises administering to a subject in need thereof an oral formulation comprising a mescaline salt, wherein the oral formulation comprises from 100-700 mg of the mescaline salt and following said administering with a mescaline treatment holiday of 2 weeks to 12 months.

In some embodiments, the mescaline treatment holiday is from 2 weeks to 12 months. In some embodiments, the mescaline treatment holiday of from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 3 weeks to 12 months. In some embodiments, the mescaline treatment holiday is from 4 weeks to 12 months. In some embodiments, the mescaline treatment holiday is from 1 month to 12 months. In some embodiments, the mescaline treatment holiday is from 3 months to 12 months. In some embodiments, the mescaline treatment holiday is from 4 months to 12 months. In some embodiments, the mescaline treatment holiday is from 5 months to 12 months. In some embodiments, the mescaline treatment holiday is from 6 months to 12 months. In some embodiments, the mescaline treatment holiday is from 7 months to 12 months. In some embodiments, the mescaline treatment holiday is from 8 months to 12 months. In some embodiments, the mescaline treatment holiday is from 9 months to 12 months. In some embodiments, the mescaline treatment holiday is from 10 months to 12 months. In some embodiments, the mescaline treatment holiday is from 11 months to 12 months. In some embodiments, the mescaline treatment holiday is 2 week, 3 weeks, 4 weeks, 1 month, 2 months, 3 months, 4 months, 5 months, 6 months, 7 months, 8 months, 9 months, 10 months, 11 months, or 12 months.

In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalants use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism.

In some embodiments, the oral formulation comprises an acid addition salt of mescaline selected from the HCl addition salt and sulfuric acid addition salt. In some embodiments, the oral formulation comprises mescaline HCl.

In some embodiments, the oral formulation comprises from 50-700 mg of mescaline or salt thereof. In some embodiments, the oral formulation comprises from about 100-600 mg of mescaline or salt thereof. In some embodiments, the oral formulation comprises from about 50-500 mg of mescaline or salt thereof. In some embodiments, the oral formulation comprises from about 50-400 mg of mescaline or salt thereof. In some embodiments, the oral formulation comprises from about 50-300 mg of mescaline or salt thereof. In some embodiments, the oral formulation comprises from about 50-200 mg of mescaline or salt thereof. In some embodiments, the oral formulation comprises from about 50-700 mg of mescaline or salt thereof. In some embodiments, the oral formulation comprises from about 50-700 mg of mescaline or salt thereof. In some embodiments, the oral formulation comprises from about 50-700 mg of mescaline or salt thereof. In some embodiments, the oral formulation comprises from about 50-700 mg of mescaline or salt thereof. In some embodiments, the oral formulation comprises from about 50-700 mg of mescaline or salt thereof. In some embodiments, the oral formulation comprises about 50 mg, about 100 mg, about 200 mg, about 300 mg, about 400 mg, about 500 mg, about 600 mg, or about 700 mg of mescaline or a salt thereof. In some embodiments, the oral formulation comprises at least 50 mg, at least 100 mg, at least 200 mg, at least 300 mg, at least 400 mg, at least 500 mg, at least 600 mg, or at least 700 mg of mescaline or a salt thereof. In some embodiments, the oral formulation comprises at most 50 mg, at most 100 mg, at most 200 mg, at most 300 mg, at most 400 mg, at most 500 mg, at most 600 mg, or at most 700 mg of mescaline or a salt thereof

In some embodiments, the method further comprises a therapy session after the administration of the oral formulation. In some embodiments, the therapy session comprises a practitioner and a plurality of subjects, wherein the practitioner abstains from the oral formulation comprising mescaline or a salt thereof. In some embodiments, the practitioner is a trained medical professional. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

In some embodiments, administering said treatment attenuates substance intake by said subject by about 20% or more as compared with the amount of substance intake before said treatment. In some embodiments, the administering said treatment attenuates substance intake by said subject, by about 30%, about 40%, about 50%, about 60%, about 70%, about 80%, about 90%, or about 95% or more as compared with the amount of substance intake before said treatment. In some embodiments, the administering said treatment attenuates substance intake by said subject, by at least 20%, at least 30%, at least 40%, at least 50%, at least 60%, at least 70%, at least 80%, at least 90%, or at least 95% as compared with the amount of substance intake before said treatment.

In some embodiments, said subject has reduced substance consumption for at least 1, 2, 3, 4, 5, 6, or 7 days. In some embodiments, said subject has reduced substance consumption for more than 1, 2, 3, 4, 5, 6, or 7 days. In some embodiments, said subject has reduced substance consumption for at least 1 day. In some embodiments, said subject has reduced substance consumption for at least 2 days. In some embodiments, said subject has reduced substance consumption for at least 3 days. In some embodiments, said subject has reduced substance consumption for at least 4 days. In some embodiments, said subject has reduced substance consumption for at least 5 days. In some embodiments, said subject has reduced substance consumption for at least 6 days. In some embodiments, said subject has reduced substance consumption for at least 7 days. In some embodiments, said subject has reduced substance consumption for more than 1 day. In some embodiments, said subject has reduced substance consumption for more than 2 days. In some embodiments, said subject has reduced substance consumption for more than 3 days. In some embodiments, said subject has reduced substance consumption for more than 4 days. In some embodiments, said subject has reduced substance consumption for more than 5 days. In some embodiments, said subject has reduced substance consumption for more than 6 days. In some embodiments, said subject has reduced substance consumption for more than 7 days.

In some embodiments, the subject has reduced substance consumption for 5-12 days prior to treatment. In some embodiments, the subject has reduced substance consumption for 5-11 days prior to treatment. In some embodiments, the subject has reduced substance consumption for 5-10 days prior to treatment. In some embodiments, the subject has reduced substance consumption for 5-9 days prior to treatment. In some embodiments, the subject has reduced substance consumption for 5-8 days prior to treatment. In some embodiments, the subject has reduced substance consumption for 5-7 days prior to treatment. In some embodiments, the subject has reduced substance consumption for 5-6 days prior to treatment. In some embodiments, the subject has reduced substance consumption for 6-12 days prior to treatment. In some embodiments, the subject has reduced substance consumption for 7-12 days prior to treatment. In some embodiments, the subject has reduced substance consumption for 8-12 days prior to treatment. In some embodiments, the subject has reduced substance consumption for 9-12 days prior to treatment. In some embodiments, the subject has reduced substance consumption for 10-12 days prior to treatment. In some embodiments, the subject has reduced substance consumption for 11-12 days prior to treatment. In some embodiments, the subject has reduced substance consumption for 1-12 days prior to treatment. In some embodiments, the subject has reduced substance consumption for 1-5 days prior to treatment. In some embodiments, the subject has reduced substance consumption for 1-4 days prior to treatment. In some embodiments, the subject has reduced substance consumption for 1-3 days prior to treatment. In some embodiments, the subject has reduced substance consumption for 1-2 days prior to treatment.

In some embodiments, the oral formulation comprises less than 5% impurities, wherein the 5% impurities are the impurities as disclosed herein.

### Treating Substance Use Disorders Using a Mescaline Drug Substance

In some aspects the present disclosure provides methods of treating substance use disorders using a mescaline drug substance. In some embodiments, the mescaline drug substance is synthetic mescaline. In some embodiments, the mescaline drug substance is a composition of Formula (VI) or Formula (IV-a), as disclosed herein. In another embodiments, the present disclosure provides a method treating a substance use disorder, comprising orally administering a pharmaceutical composition comprising a mescaline drug substance and a pharmaceutically acceptable excipient to the subject and accompanying said administration with a therapy session, wherein the pharmaceutical composition comprises less than 5% impurities.

In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalants use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the mescaline drug substance comprises less than 5% impurities. In some embodiments, the mescaline drug substance comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the mescaline drug substance comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from plant cells, plant tissue, and naturally occurring alkaloids. In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by:

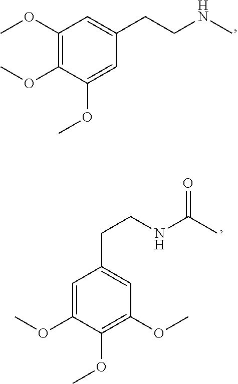

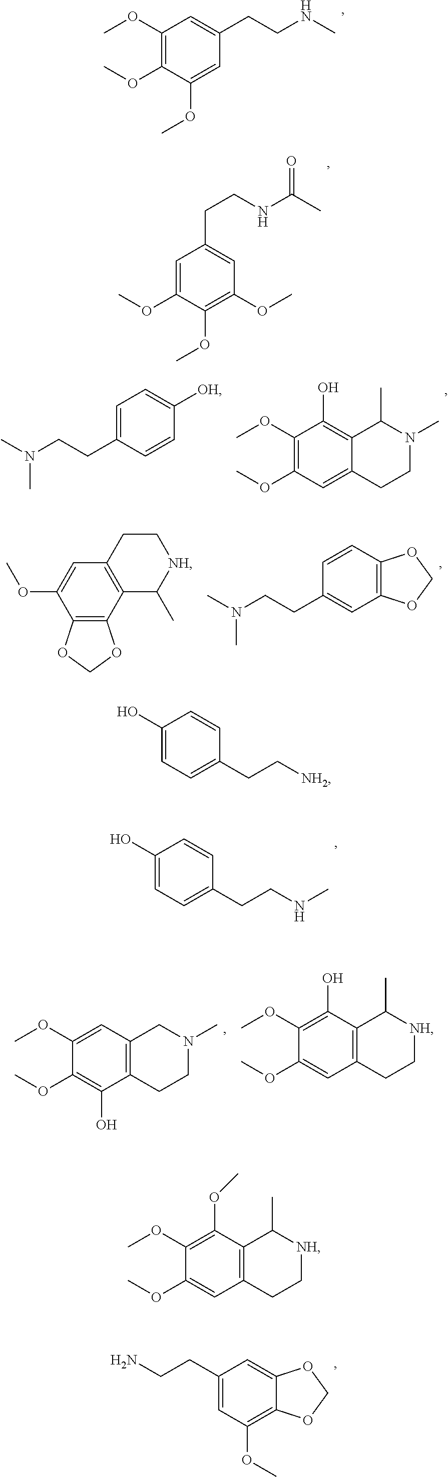

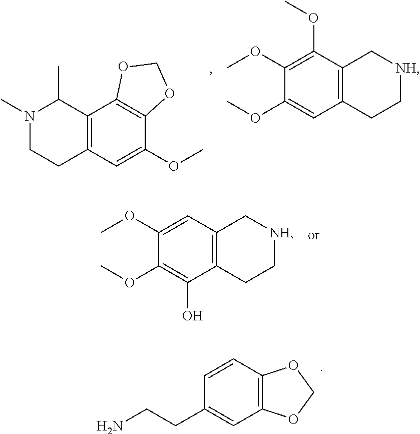

In some embodiments, the method further comprises a mescaline treatment holiday of from 2 weeks to 12 months. In some embodiments, the mescaline treatment holiday is from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 2 weeks to 2 months. In some embodiments, the mescaline treatment holiday is from 2 weeks to 1 months. In some embodiments, the mescaline treatment holiday is from 2 weeks to 4 weeks. In some embodiments, the mescaline treatment holiday is from 2 weeks to 3 weeks. In some embodiments, the mescaline treatment holiday is from 1 month, 2 months, 3 months, 4, month, 5 months, 6 months, 7 month, 8 months, 9 months, 10 months, 11 months, or 12 months. In some embodiments, the mescaline treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the therapy session comprises a practitioner and a plurality of subjects, wherein the practitioner abstains from the oral administration of the pharmaceutical composition comprising mescaline or a salt thereof. In some embodiments, the practitioner is a trained medical professional. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

### Treating Substance Use Disorders Based on a Treatment Plan

In certain aspects the present disclosure provides methods of treating substance use disorders based multiple treatment steps. In another embodiments, the present disclosure provides a method of treating an substance use disorder comprising (a) administering a first treatment comprising mescaline or a salt thereof and accompanying said first treatment with a first therapy session; (b) administering a second treatment 5-12 days after said first treatment, said second treatment comprising mescaline or a salt thereof and accompanying said second treatment with a second therapy session.

In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalants use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism.

In some embodiments, the method further comprises one or more maintenance treatments comprising mescaline or a salt thereof and accompanying said maintenance treatment with a maintenance therapy session. In some embodiments, the one or more maintenance treatments comprise administering a one or more maintenance treatments comprising mescaline or a salt thereof and accompanying said one or more maintenance treatments with a one or more maintenance therapy session.

In some embodiments, the first therapy session, the second therapy session, and the maintenance therapy session comprises a practitioner and a plurality of subjects, wherein the practitioner abstains from the administration comprising mescaline or a salt thereof. In some embodiments, the first therapy session, the second therapy session, and the maintenance therapy session comprises the practitioner and at least one subject. In some embodiments, the first therapy session, the second therapy session, and the maintenance therapy session comprises psychosocial therapy.

In some embodiments, the first therapy session, comprises a practitioner and a plurality of subjects, wherein the practitioner abstains from the administration comprising mescaline or a salt thereof. In some embodiments, the practitioner is a trained medical professional. In some embodiments, the first therapy session comprises a practitioner and a plurality of subjects. In some embodiments, the first therapy session comprises the practitioner and at least one subject. In some embodiments, the first therapy session is a group session with the plurality of subjects. In some embodiments, the first group session comprises the practitioner and at least 2 subjects. In some embodiments, the first therapy session comprises psychosocial therapy.

In some embodiments, the second therapy session, comprises a practitioner and a plurality of subjects, wherein the practitioner abstains from the administration comprising mescaline or a salt thereof. In some embodiments, the practitioner is a trained medical professional. In some embodiments, the second therapy session comprises a practitioner and a plurality of subjects. In some embodiments, the second therapy session comprises the practitioner and at least one subject. In some embodiments, the second therapy session is a group session with the plurality of subjects. In some embodiments, the second group session comprises the practitioner and at least 2 subjects. In some embodiments, the second therapy session comprises psychosocial therapy.

In some embodiments, the maintenance therapy session, comprises a practitioner and a plurality of subjects, wherein the practitioner abstains from the administration comprising mescaline or a salt thereof. In some embodiments, the practitioner is a trained medical professional. In some embodiments, the maintenance therapy session comprises a practitioner and a plurality of subjects. In some embodiments, the maintenance therapy session comprises the practitioner and at least one subject. In some embodiments, the maintenance therapy session is a group session with the plurality of subjects. In some embodiments, the maintenance group session comprises the practitioner and at least 2 subjects. In some embodiments, the maintenance therapy session comprises psychosocial therapy.

In some embodiments, the method further comprises administering a preparatory therapy session before said first therapy session. In some embodiments, the preparatory therapy session involves abstaining from the administration of mescaline or a salt thereof. In some embodiments, the preparatory therapy session comprises psychosocial therapy. In some embodiments, the preparatory therapy session, comprises a practitioner and a plurality of subjects. In some embodiments, the practitioner is a trained medical professional. In some embodiments, the preparatory session comprises a practitioner and a plurality of subjects. In some embodiments, the preparatory therapy session comprises the practitioner and at least one subject. In some embodiments, the preparatory therapy session is a group session with the plurality of subjects. In some embodiments, the preparatory group session comprises the practitioner and at least 2 subjects. In some embodiments, the preparatory group session comprises the practitioner and a subject.

In some embodiments, the mescaline or a salt thereof is administered as a pharmaceutical composition comprising mescaline or a salt thereof and a pharmaceutically acceptable excipient, wherein the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities. In some embodiments, the impurity is selected from plant cells, plant tissue, and naturally occurring alkaloids.

In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by:

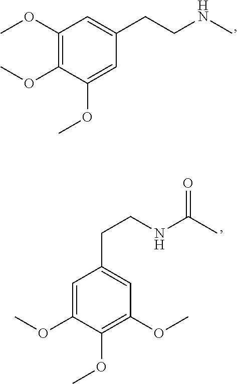

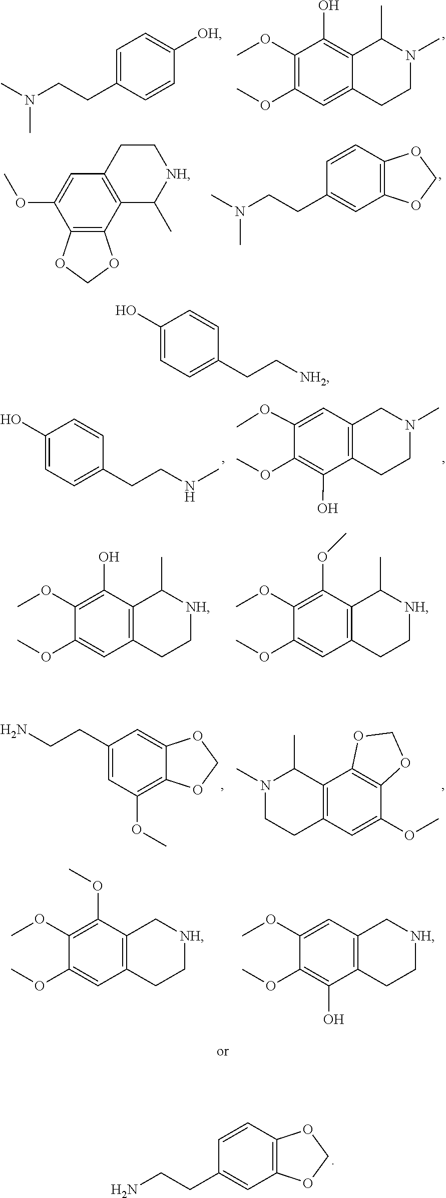

In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form. In some embodiments, the pharmaceutical composition in the tablet comprises less than 5% impurities as disclosed herein. In some embodiments, the pharmaceutical composition in the capsule comprises less than 5% impurities as disclosed herein.

In some embodiments, 80% or more of the pharmaceutical composition comprises mescaline or salt thereof. In some embodiments, 90% or more of the pharmaceutical composition comprises mescaline or salt thereof. In some embodiments, 95% or more of the pharmaceutical composition comprises mescaline or salt thereof. In some embodiments, 80% or more, 85% or more, 90% or more, 95% or more, or 99% or more of the pharmaceutical composition comprises mescaline or salt thereof. In some embodiments, at least 80%, at least 85%, at least 90%, at least 95%, or at least 99% of the pharmaceutical composition comprises mescaline or salt thereof.

In some embodiments, the method further comprises is identifying the subject with a substance use disorder by a medical professional using symptoms or criteria as defined by DSM-5. In some embodiments, the substance use disorder in a subject is diagnosed by a medical professional. In some embodiments, the substance use disorder in a subject is diagnosed as a mild substance use disorder. In some embodiments, the substance use disorder in a subject is diagnosed as a moderate substance use disorder. In some embodiments, the substance use disorder in a subject is diagnosed as a severe substance use disorders.

### (b) Methods of Treating Alcohol Use Disorders

### Treating Alcohol Use Disorder Based on Reduced Alcohol Consumption

In certain aspects the present disclosure provides methods of treating alcohol use disorders. In some embodiments, the method for treating an alcohol use disorder comprises (a) identifying a subject who has an alcohol use disorder and who has reduced alcohol consumption for at least 1 days prior to the administering of step (b); and (b) orally administering a pharmaceutical composition comprising mescaline or a salt thereof to the subject and accompanying said administration with a therapy session.

In some embodiments, the reduced alcohol consumption is abstaining from moderate to heavy alcohol consumption. In some embodiments, the reduced alcohol consumption is measured by a reduction in heavy drinking days. In some embodiments, the reduced alcohol consumption is measured by reduction in risk drinking level. In some embodiments, the reduced alcohol consumption is abstaining from alcohol consumption. In some embodiments, the reduced alcohol consumption is a reduction in binge drinking as defined herein. In some embodiments, the reduced alcohol consumption is abstaining from binge drinking.

In some embodiments, administering said treatment attenuates alcohol intake by said subject by about 20% or more as compared with the amount of alcohol intake before said treatment. In some embodiments, the administering said treatment attenuates alcohol intake by said subject by about 30%, about 40%, about 50%, about 60%, about 70%, about 80%, about 90%, or about 95% or more as compared with the amount of alcohol intake before said treatment. In some embodiments, the administering said treatment attenuates alcohol intake by said subject by at least 20%, at least 30%, at least 40%, at least 50%, at least 60%, at least 70%, at least 80%, at least 90%, or at least 95% as compared with the amount of alcohol intake before said treatment.

In some embodiments, the alcohol use disorder is selected from alcohol abuse, alcohol dependence, and alcoholism. In some embodiments, the alcohol use disorder is selected from alcohol abuse. In some embodiments, the alcohol use disorder is selected from alcohol dependence. In some embodiments, the alcohol use disorder is selected from alcoholism.

In some embodiments, for step (a), said subject has reduced alcohol consumption for at least 1, 2, 3, 4, 5, 6, or 7 days prior to step (b). In some embodiments, for step (a), said subject has reduced alcohol consumption for at least 3, 4, 5, 6, or 7 days prior to step (b). In some embodiments, for step (a), said subject has reduced alcohol consumption for more than 1, 2, 3, 4, 5, 6, or 7 days prior to step (b). In some embodiments, for step (a), said subject has reduced alcohol consumption for more than 3, 4, 5, 6, or 7 days prior to step (b). In some embodiments, for step (a), said subject has reduced alcohol consumption for at least 2 days prior to step (b). In some embodiments, for step (a), said subject has reduced alcohol consumption for at least 3 days prior to step (b). In some embodiments, for step (a), said subject has reduced alcohol consumption for at least 4 days prior to step (b). In some embodiments, for step (a), said subject has reduced alcohol consumption for at least 5 days prior to step (b). In some embodiments, for step (a), said subject has reduced alcohol consumption for at least 6 days prior to step (b). In some embodiments, for step (a), said subject has reduced alcohol consumption for at least 7 days prior to step (b). In some embodiments, for step (a), said subject has reduced alcohol consumption for more than 2 days prior to step (b). In some embodiments, for step (a), said subject has reduced alcohol consumption for more than 3 days prior to step (b). In some embodiments, for step (a), said subject has reduced alcohol consumption for more than 4 days prior to step (b). In some embodiments, for step (a), said subject has reduced alcohol consumption for more than 5 days prior to step (b). In some embodiments, for step (a), said subject has reduced alcohol consumption for more than 6 days prior to step (b). In some embodiments, for step (a), said subject has reduced alcohol consumption for more than 7 days prior to step (b).

In some embodiments, the subject has reduced alcohol consumption for 1-12 days. In some embodiments, the subject has reduced alcohol consumption for 2-12 days. In some embodiments, the subject has reduced alcohol consumption for 3-12 days. In some embodiments, the subject has reduced alcohol consumption for 4-12 days. In some embodiments, the subject has reduced alcohol consumption for 5-12 days. In some embodiments, the subject has reduced alcohol consumption for 5-11 days. In some embodiments, the subject has reduced alcohol consumption for 5-10 days. In some embodiments, the subject has reduced alcohol consumption for 5-9 days. In some embodiments, the subject has reduced alcohol consumption for 5-8 days. In some embodiments, the subject has reduced alcohol consumption for 5-7 days. In some embodiments, the subject has reduced alcohol consumption for 5-6 days. In some embodiments, the subject has reduced alcohol consumption for 6-12 days. In some embodiments, the subject has reduced alcohol consumption for 7-12 days. In some embodiments, the subject has reduced alcohol consumption for 8-12 days. In some embodiments, the subject has reduced alcohol consumption for 9-12 days. In some embodiments, the subject has reduced alcohol consumption for 10-12 days. In some embodiments, the subject has reduced alcohol consumption for 11-12 days.

In some embodiments, further comprising a mescaline treatment holiday of from 2 weeks to 12 months. In some embodiments, the mescaline treatment holiday is from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 2 weeks to 2 months. In some embodiments, the mescaline treatment holiday is from 2 weeks to 1 months. In some embodiments, the mescaline treatment holiday is from 2 weeks to 4 weeks. In some embodiments, the mescaline treatment holiday is from 2 weeks to 3 weeks. In some embodiments, the mescaline treatment holiday is from 1 month, 2 months, 3 months, 4, month, 5 months, 6 months, 7 month, 8 months, 9 months, 10 months, 11 months, or 12 months. In some embodiments, the mescaline treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the pharmaceutical composition comprises an acid addition salt of mescaline selected from the HCl addition salt and sulfuric acid addition salt. In some embodiments, the pharmaceutical composition comprises mescaline HCl.

In some embodiments, the pharmaceutical composition comprises from 50-700 mg of mescaline or salt thereof. In some embodiments, the pharmaceutical composition comprises from about 50-600 mg of mescaline or salt thereof. In some embodiments, the pharmaceutical composition comprises from about 50-500 mg of mescaline or salt thereof. In some embodiments, the pharmaceutical composition comprises from about 50-400 mg of mescaline or salt thereof. In some embodiments, the pharmaceutical composition comprises from about 50-300 mg of mescaline or salt thereof. In some embodiments, the pharmaceutical composition comprises from about 50-200 mg of mescaline or salt thereof. In some embodiments, the pharmaceutical composition comprises from about 200-700 mg of mescaline or salt thereof. In some embodiments, the pharmaceutical composition comprises from about 300-700 mg of mescaline or salt thereof. In some embodiments, the pharmaceutical composition comprises from about 400-700 mg of mescaline or salt thereof. In some embodiments, the pharmaceutical composition comprises from about 500-700 mg of mescaline or salt thereof. In some embodiments, the pharmaceutical composition comprises from about 600-700 mg of mescaline or salt thereof. In some embodiments, the pharmaceutical composition comprises about 50 mg, about 100 mg, about 200 mg, about 300 mg, about 400 mg, about 500 mg, about 600 mg, or about 700 mg of mescaline or a salt thereof. In some embodiments, the pharmaceutical composition comprises at least 50 mg, at least 100 mg, at least 200 mg, at least 300 mg, at least 400 mg, at least 500 mg, at least 600 mg, or at least 700 mg of mescaline or a salt thereof. In some embodiments, the pharmaceutical composition comprises at most 50 mg, at most 100 mg, at most 200 mg, at most 300 mg, at most 400 mg, at most 500 mg, at most 600 mg, or at most 700 mg of mescaline or a salt thereof.

In some embodiments, the therapy session comprises a practitioner and a plurality of subjects, wherein the practitioner abstains from the oral administration of the pharmaceutical composition comprising mescaline or a salt thereof. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialists, a substance abuse counselor, a therapist, or any combination thereof. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities. In some embodiments, the impurity is selected from plant cells, plant tissue, and naturally occurring alkaloids.

In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by

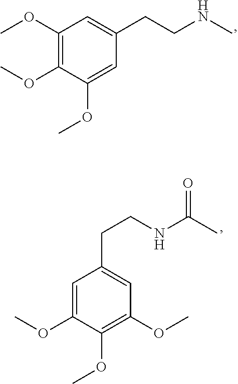

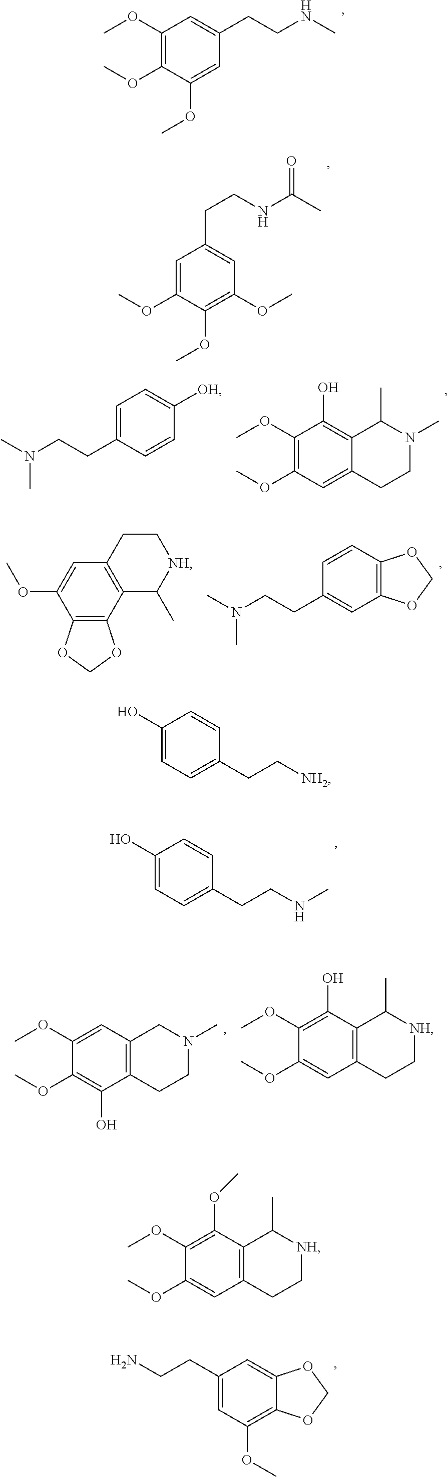

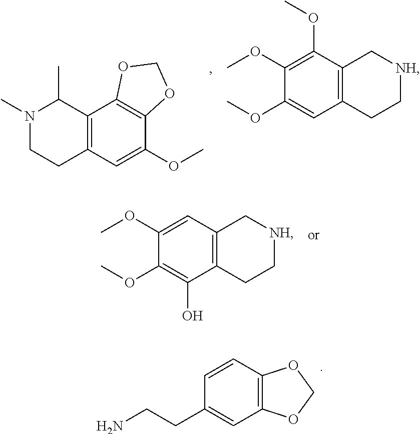

In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form. In some embodiments, the pharmaceutical composition in the tablet comprises less than 5% impurities as disclosed herein. In some embodiments, the pharmaceutical composition in the capsule comprises less than 5% impurities as disclosed herein.

In some embodiments, 80% or more of the pharmaceutical composition comprises mescaline or salt thereof. In some embodiments, 90% or more of the pharmaceutical composition comprises mescaline or salt thereof. In some embodiments, 95% or more of the pharmaceutical composition comprises mescaline or salt thereof. In some embodiments, 80% or more, 85% or more, 90% or more, 95% or more, or 99% or more of the pharmaceutical composition comprises mescaline or salt thereof. In some embodiments, at least 80%, at least 85%, at least 90%, at least 95%, or at least 99% of the pharmaceutical composition comprises mescaline or salt thereof.

### Treating Alcohol Use Disorder Based on Mescaline Treatment Holiday

In some aspects the present disclosure provides methods of treating alcohol use disorders based on a designated mescaline drug holiday. In another embodiments, the method of treating an alcohol use disorder comprises administering to a subject in need thereof an oral formulation comprising a mescaline salt, wherein the oral formulation comprises from 100-700 mg of the mescaline salt and following said administering with a mescaline treatment holiday of 2 weeks to 12 months.

In some embodiments, the mescaline treatment holiday is from 2 weeks to 12 months. In some embodiments, the mescaline treatment holiday of from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 3 weeks to 12 months. In some embodiments, the mescaline treatment holiday is from 4 weeks to 12 months. In some embodiments, the mescaline treatment holiday is from 1 month to 12 months. In some embodiments, the mescaline treatment holiday is from 3 months to 12 months. In some embodiments, the mescaline treatment holiday is from 4 months to 12 months. In some embodiments, the mescaline treatment holiday is from 5 months to 12 months. In some embodiments, the mescaline treatment holiday is from 6 months to 12 months. In some embodiments, the mescaline treatment holiday is from 7 months to 12 months. In some embodiments, the mescaline treatment holiday is from 8 months to 12 months. In some embodiments, the mescaline treatment holiday is from 9 months to 12 months. In some embodiments, the mescaline treatment holiday is from 10 months to 12 months. In some embodiments, the mescaline treatment holiday is from 11 months to 12 months. In some embodiments, the mescaline treatment holiday is 2 week, 3 weeks, 4 weeks, 1 month, 2 months, 3 months, 4 months, 5 months, 6 months, 7 months, 8 months, 9 months, 10 months, 11 months, or 12 months.

In some embodiments, the oral formulation comprises an acid addition salt of mescaline selected from the HCl addition salt and sulfuric acid addition salt. In some embodiments, the oral formulation comprises mescaline HCl.

In some embodiments, the oral formulation comprises from 50-700 mg of mescaline or salt thereof. In some embodiments, the oral formulation comprises from about 50-600 mg of mescaline or salt thereof. In some embodiments, the oral formulation comprises from about 50-500 mg of mescaline or salt thereof. In some embodiments, the oral formulation comprises from about 50-400 mg of mescaline or salt thereof. In some embodiments, the oral formulation comprises from about 50-300 mg of mescaline or salt thereof. In some embodiments, the oral formulation comprises from about 50-200 mg of mescaline or salt thereof. In some embodiments, the oral formulation comprises from about 200-700 mg of mescaline or salt thereof. In some embodiments, the oral formulation comprises from about 300-700 mg of mescaline or salt thereof. In some embodiments, the oral formulation comprises from about 400-700 mg of mescaline or salt thereof. In some embodiments, the oral formulation comprises from about 500-700 mg of mescaline or salt thereof. In some embodiments, the oral formulation comprises from about 600-700 mg of mescaline or salt thereof. In some embodiments, the oral formulation comprises about 50 mg, about 100 mg, about 200 mg, about 300 mg, about 400 mg, about 500 mg, about 600 mg, or about 700 mg of mescaline or a salt thereof. In some embodiments, the oral formulation comprises at least 50 mg, at least 100 mg, at least 200 mg, at least 300 mg, at least 400 mg, at least 500 mg, at least 600 mg, or at least 700 mg of mescaline or a salt thereof. In some embodiments, the oral formulation comprises at most 50 mg, at most 100 mg, at most 200 mg, at most 300 mg, at most 400 mg, at most 500 mg, at most 600 mg, or at most 700 mg of mescaline or a salt thereof

In some embodiments, the method further comprises a therapy session after the administration of the oral formulation. In some embodiments, the therapy session comprises a practitioner and a plurality of subjects, wherein the practitioner abstains from the oral formulation comprising mescaline or a salt thereof. In some embodiments, the practitioner is a trained medical professional. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

In some embodiments, administering said treatment attenuates alcohol intake by said subject by about 20% or more as compared with the amount of alcohol intake before said treatment. In some embodiments, the administering said treatment attenuates alcohol intake by said subject by about 30%, about 40%, about 50%, about 60%, about 70%, about 80%, about 90%, or about 95% or more as compared with the amount of alcohol intake before said treatment. In some embodiments, the administering said treatment attenuates alcohol intake by said subject by at least 20%, at least 30%, at least 40%, at least 50%, at least 60%, at least 70%, at least 80%, at least 90%, or at least 95% as compared with the amount of alcohol intake before said treatment.

In some embodiments, the alcohol use disorder is selected from alcohol abuse, alcohol dependence, and alcoholism. In some embodiments, the alcohol use disorder is selected from alcohol abuse. In some embodiments, the alcohol use disorder is selected from alcohol dependence. In some embodiments, the alcohol use disorder is selected from alcohol dependence. In some embodiments, the alcohol use disorder is a substance use disorder as defined under DSM-5.

In some embodiments, said subject has reduced alcohol consumption for at least 1, 2, 3, 4, 5, 6, or 7 days. In some embodiments, said subject has reduced alcohol consumption for more than 1, 2, 3, 4, 5, 6, or 7 days. In some embodiments, said subject has reduced alcohol consumption for at least 1 day. In some embodiments, said subject has reduced alcohol consumption for at least 2 days. In some embodiments, said subject has reduced alcohol consumption for at least 3 days. In some embodiments, said subject has reduced alcohol consumption for at least 4 days. In some embodiments, said subject has reduced alcohol consumption for at least 5 days. In some embodiments, said subject has reduced alcohol consumption for at least 6 days. In some embodiments, said subject has reduced alcohol consumption for at least 7 days. In some embodiments, said subject has reduced alcohol consumption for more than 1 day. In some embodiments, said subject has reduced alcohol consumption for more than 2 days. In some embodiments, said subject has reduced alcohol consumption for more than 3 days. In some embodiments, said subject has reduced alcohol consumption for more than 4 days. In some embodiments, said subject has reduced alcohol consumption for more than 5 days. In some embodiments, said subject has reduced alcohol consumption for more than 6 days. In some embodiments, said subject has reduced alcohol consumption for more than 7 days.

In some embodiments, the subject has reduced alcohol consumption for 5-12 days prior to treatment. In some embodiments, the subject has reduced alcohol consumption for 5-11 days prior to treatment. In some embodiments, the subject has reduced alcohol consumption for 5-10 days prior to treatment. In some embodiments, the subject has reduced alcohol consumption for 5-9 days prior to treatment. In some embodiments, the subject has reduced alcohol consumption for 5-8 days prior to treatment. In some embodiments, the subject has reduced alcohol consumption for 5-7 days prior to treatment. In some embodiments, the subject has reduced alcohol consumption for 5-6 days prior to treatment. In some embodiments, the subject has reduced alcohol consumption for 6-12 days prior to treatment. In some embodiments, the subject has reduced alcohol consumption for 7-12 days prior to treatment. In some embodiments, the subject has reduced alcohol consumption for 8-12 days prior to treatment. In some embodiments, the subject has reduced alcohol consumption for 9-12 days prior to treatment. In some embodiments, the subject has reduced alcohol consumption for 10-12 days prior to treatment. In some embodiments, the subject has reduced alcohol consumption for 11-12 days prior to treatment. In some embodiments, the subject has reduced alcohol consumption for 1-12 days prior to treatment. In some embodiments, the subject has reduced alcohol consumption for 1-5 days prior to treatment. In some embodiments, the subject has reduced alcohol consumption for 1-4 days prior to treatment. In some embodiments, the subject has reduced alcohol consumption for 1-3 days prior to treatment. In some embodiments, the subject has reduced alcohol consumption for 1-2 days prior to treatment.

In some embodiments, the oral formulation comprises less than 5% impurities, wherein the 5% impurities are the impurities as disclosed herein.

### Treating Alcohol Use Disorder Using a Mescaline Drag Substance

In some aspects the present disclosure provides methods of treating alcohol use disorders using a mescaline drug substance. In another embodiments, the present disclosure provides a method treating an alcohol use disorder, comprising orally administering a pharmaceutical composition comprising a mescaline drug substance and a pharmaceutically acceptable excipient to the subject and accompanying said administration with a therapy session, wherein the pharmaceutical composition comprises less than 5% impurities.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the mescaline drug substance comprises less than 5% impurities. In some embodiments, the mescaline drug substance comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the mescaline drug substance comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from plant cells, plant tissue, and naturally occurring alkaloids. In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by:

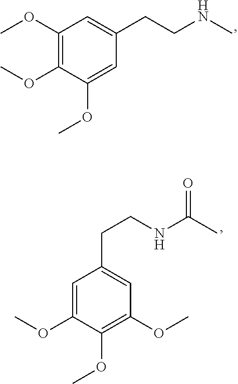

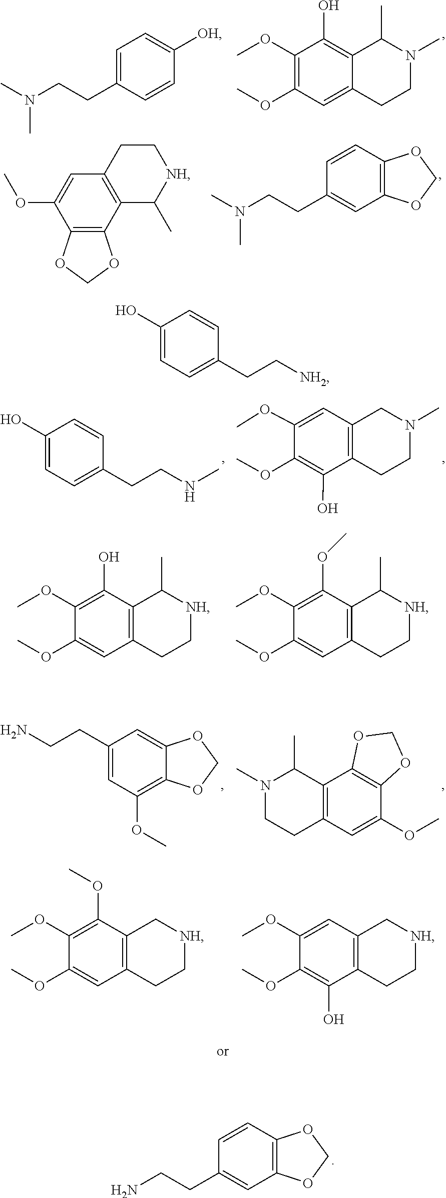

In some embodiments, the method further comprises a mescaline treatment holiday of from 2 weeks to 12 months. In some embodiments, the mescaline treatment holiday is from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 2 weeks to 2 months. In some embodiments, the mescaline treatment holiday is from 2 weeks to 1 months. In some embodiments, the mescaline treatment holiday is from 2 weeks to 4 weeks. In some embodiments, the mescaline treatment holiday is from 2 weeks to 3 weeks. In some embodiments, the mescaline treatment holiday is from 1 month, 2 months, 3 months, 4, month, 5 months, 6 months, 7 month, 8 months, 9 months, 10 months, 11 months, or 12 months. In some embodiments, the mescaline treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the therapy session comprises a practitioner and a plurality of subjects, wherein the practitioner abstains from the oral administration of the pharmaceutical composition comprising mescaline or a salt thereof. In some embodiments, the practitioner is a trained medical professional. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

### Treating Alcohol Use Disorder Based on a Treatment Plan

In certain aspects the present disclosure provides methods of treating alcohol use disorders based multiple treatment steps. In another embodiments, the present disclosure provides a method of treating an alcohol use disorder comprising (a) administering a first treatment comprising mescaline or a salt thereof and accompanying said first treatment with a first therapy session; (b) administering a second treatment 5-12 days after said first treatment, said second treatment comprising mescaline or a salt thereof and accompanying said second treatment with a second therapy session.

In some embodiments, the method further comprises one or more maintenance treatments comprising mescaline or a salt thereof and accompanying said maintenance treatment with a maintenance therapy session. In some embodiments, the one or more maintenance treatments comprise administering a one or more maintenance treatments comprising mescaline or a salt thereof and accompanying said one or more maintenance treatments with a one or more maintenance therapy session.

In some embodiments, the first therapy session, the second therapy session, and the maintenance therapy session comprises a practitioner and a plurality of subjects, wherein the practitioner abstains from the administration comprising mescaline or a salt thereof. In some embodiments, the first therapy session, the second therapy session, and the maintenance therapy session comprises the practitioner and at least one subject. In some embodiments, the first therapy session, the second therapy session, and the maintenance therapy session comprises psychosocial therapy.

In some embodiments, the first therapy session, comprises a practitioner and a plurality of subjects, wherein the practitioner abstains from the administration comprising mescaline or a salt thereof. In some embodiments, the practitioner is a trained medical professional. In some embodiments, the first therapy session comprises a practitioner and a plurality of subjects. In some embodiments, the first therapy session comprises the practitioner and at least one subject. In some embodiments, the first therapy session is a group session with the plurality of subjects. In some embodiments, the first group session comprises the practitioner and at least 2 subjects. In some embodiments, the first therapy session comprises psychosocial therapy.

In some embodiments, the second therapy session, comprises a practitioner and a plurality of subjects, wherein the practitioner abstains from the administration comprising mescaline or a salt thereof. In some embodiments, the practitioner is a trained medical professional. In some embodiments, the second therapy session comprises a practitioner and a plurality of subjects. In some embodiments, the second therapy session comprises the practitioner and at least one subject. In some embodiments, the second therapy session is a group session with the plurality of subjects. In some embodiments, the second group session comprises the practitioner and at least 2 subjects. In some embodiments, the second therapy session comprises psychosocial therapy.

In some embodiments, the maintenance therapy session, comprises a practitioner and a plurality of subjects, wherein the practitioner abstains from the administration comprising mescaline or a salt thereof. In some embodiments, the practitioner is a trained medical professional. In some embodiments, the maintenance therapy session comprises a practitioner and a plurality of subjects. In some embodiments, the maintenance therapy session comprises the practitioner and at least one subject. In some embodiments, the maintenance therapy session is a group session with the plurality of subjects. In some embodiments, the maintenance group session comprises the practitioner and at least 2 subjects. In some embodiments, the maintenance therapy session comprises psychosocial therapy.

In some embodiments, the method further comprises administering a preparatory therapy session before said first therapy session. In some embodiments, the preparatory therapy session involves abstaining from the administration of mescaline or a salt thereof. In some embodiments, the preparatory therapy session comprises psychosocial therapy. In some embodiments, the preparatory therapy session, comprises a practitioner and a plurality of subjects. In some embodiments, the practitioner is a trained medical professional. In some embodiments, the preparatory session comprises a practitioner and a plurality of subjects. In some embodiments, the preparatory therapy session comprises the practitioner and at least one subject. In some embodiments, the preparatory therapy session is a group session with the plurality of subjects. In some embodiments, the preparatory group session comprises the practitioner and at least 2 subjects. In some embodiments, the preparatory group session comprises the practitioner and a subject.

In some embodiments, the mescaline or a salt thereof is administered as a pharmaceutical composition comprising mescaline or a salt thereof and a pharmaceutically acceptable excipient, wherein the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities. In some embodiments, the impurity is selected from plant cells, plant tissue, and naturally occurring alkaloids.

In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the impurity is selected from an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by:

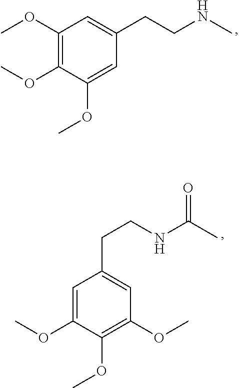

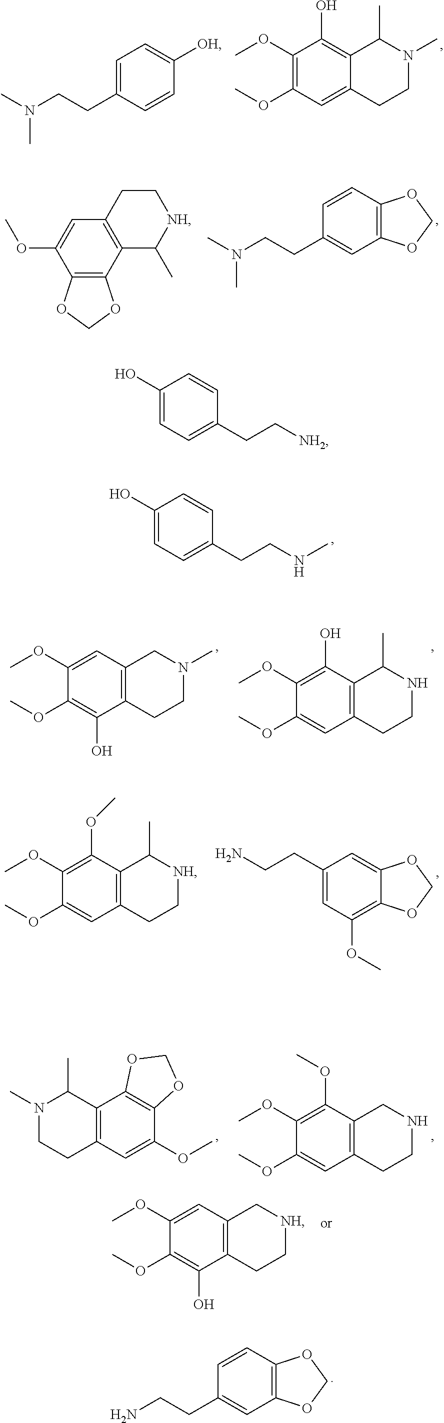

In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form. In some embodiments, the pharmaceutical composition in the tablet comprises less than 5% impurities as disclosed herein. In some embodiments, the pharmaceutical composition in the capsule comprises less than 5% impurities as disclosed herein.

In some embodiments, 80% or more of the pharmaceutical composition comprises mescaline or salt thereof. In some embodiments, 90% or more of the pharmaceutical composition comprises mescaline or salt thereof. In some embodiments, 95% or more of the pharmaceutical composition comprises mescaline or salt thereof. In some embodiments, 80% or more, 85% or more, 90% or more, 95% or more, or 99% or more of the pharmaceutical composition comprises mescaline or salt thereof. In some embodiments, at least 80%, at least 85%, at least 90%, at least 95%, or at least 99% of the pharmaceutical composition comprises mescaline or salt thereof.

### (c) Methods of Producing Synthetic and Pure Forms of Mescaline

In certain aspects the present disclosure provides methods of synthesizing a compound of Formula (IV).

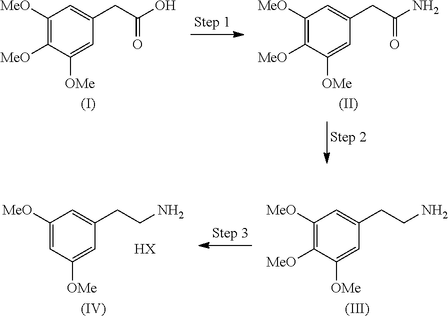

In some embodiments, the present disclosure provides methods of producing a compound of Formula (II). In some embodiments, the present disclosure provides methods of producing a compound of Formula (III). In some embodiments, the present disclosure provides methods of producing a compound of Formula (IV). In some embodiments, the method provided herein, is performed under Good Manufacturing Practices (GMP) conditions. In additional embodiments, the method is performed in a GMP facility.

In some embodiments, the present disclosure provides a compound of Formula (II). In some embodiments, the compound of Formula (II) comprises additional characteristics. In some embodiments, the compound of Formula (II) is produced using step 1. In some embodiments, the present disclosure provides a compound of Formula (III). In some embodiments, the compound of Formula (III) comprises additional characteristics. In some embodiments, the compound of Formula (III) is produced using step 2. In some embodiments, the present disclosure provides a compound of Formula (IV). In some embodiments, the compound of Formula (IV) comprises additional characteristics. In some embodiments, the compound of Formula (IV) is produced using step 3.

In some embodiments, the compound or salt of Formula (IV) may be formulated into a pharmaceutical composition. In some embodiments, the pharmaceutical composition is in a tablet or a capsule. In some embodiments, the pharmaceutical composition is a tablet. In some embodiments, the pharmaceutical composition is a capsule. In some embodiments, the pharmaceutical composition is in an oral dosage form.

In some embodiments, 80% or more of the pharmaceutical composition comprises the compound or salt of Formula (IV). In some embodiments, 90% or more of the pharmaceutical composition comprises the compound or salt of Formula (IV). In some embodiments, 95% or more of the pharmaceutical composition comprises the compound or salt of Formula (IV). In some embodiments, 80% or more, 85% or more, 90% or more, 95% or more, or 99% or more of the compound or salt of Formula (IV). In some embodiments, at least 80%, at least 85%, at least 90%, at least 95%, or at least 99% of the pharmaceutical composition comprises the compound or salt of Formula (IV).

The procedures described herein are useful for the production of synthetic mescaline and salts thereof. The methods described herein are advantageous over direct extraction from mescaline containing plants such as: Lophophora williamsii, Trichocereus (Echinopsis) pachanoi, Echinopsis peruviana, Trichocereus bridgesii, Pereskia aculeata, Acacia berlandieri, or Pelecyphora aselliformis. The compositions described herein are free of plant cells, plant tissue, and/or naturally occurring alkaloids. For example, the compositions disclosed herein, lack naturally occurring alkaloids associated with the extraction of mescaline from plants. In some embodiments, the naturally occurring alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the naturally occurring alkaloid is represented by:

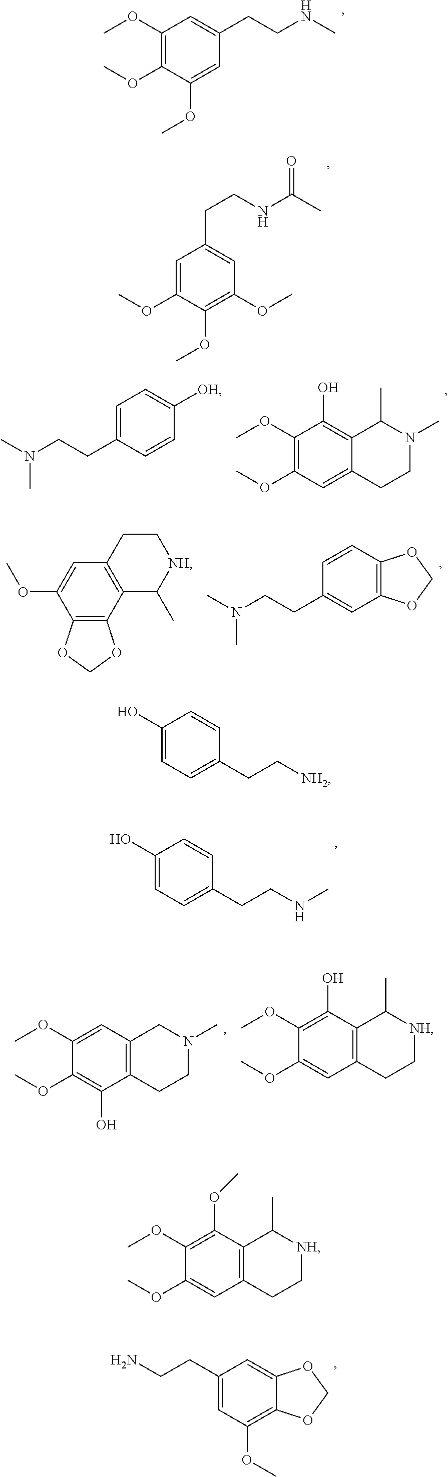

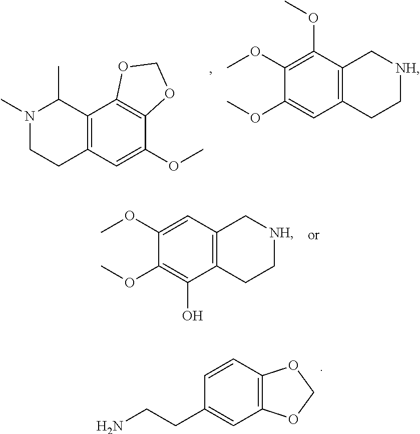

or combinations thereof.

In another aspect, the present disclosure provides a composition comprising a compound represented by Formula (II):

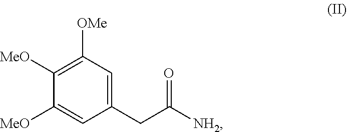 [SMILES: COc1cc(CC(N)=O)cc(OC)c1OC]

wherein the composition is characterized by one or more additional characteristics selected from: (i) between about 0.1 wt % to about 10 wt % imidazole content; (ii) between about 0.1 wt % to about 1.0 wt % dichloromethane content; and between about 0.1 wt % to about 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

In some embodiments, a composition comprising a compound represented by Formula (II):

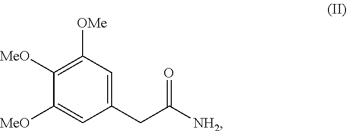 [SMILES: COc1cc(CC(N)=O)cc(OC)c1OC]

the composition is characterized by one or more additional characteristics selected from: (i) at least 0.1 wt % imidazole content; (ii) at most 1.0 wt % dichloromethane content; and at most 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

In some embodiments, a composition comprising a compound represented by Formula (II):

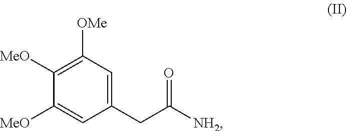 [SMILES: COc1cc(CC(N)=O)cc(OC)c1OC]

wherein the composition is characterized by one or more additional characteristics selected from: (i) between 0.1 wt % to 10 wt % imidazole content; (ii) between 0.1 wt % to 1.0 wt % dichloromethane content; and between 0.1 wt % to 3.0 wt % 3,4,5-Trimethoxyphenyl acetic acid. In some embodiments, the compositions comprising Formula (II) is characterized by at least two characteristics. In some embodiments, the compositions comprising Formula (II) is characterized by three characteristics.

In another aspect, the present disclosure provides a composition comprising a compound represented by Formula (III):

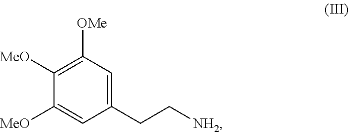

wherein, the composition is characterized by one or more additional characteristics selected from: from: (i) between about 0.1 wt % to about 8.0 wt % 2-(3,4,5-trimethoxyphenyl)acetamide; (ii) between about 0.1 wt % to about 5 wt % dimeric amine; (iii) between about 0.1 wt % to about 5 wt % n-butanol; and (iv) between about 0.1 wt % to about 6% toluene. In some embodiments, the composition is characterized by two or more characteristics selected from i to iv. In some embodiments, the composition is characterized by at least two or more characteristics selected from i to iv. In some embodiments, the composition is characterized by at least three or more characteristics selected from i to iv. In some embodiments, the composition is characterized by at least four or more characteristics selected from i to iv. In some embodiments, the composition is characterized by at least five or more characteristics selected from i to iv. In some embodiments, the composition is characterized by at least six characteristics selected from i to iv. In some embodiments, 2-(3,4,5-trimethoxyphenyl)acetamide is represented by

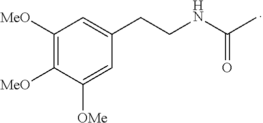 [SMILES: COc1cc(CCNC(C)=O)cc(OC)c1OC]

In some embodiments, 2-(3,4,5-trimethoxyphenyl)acetamide is between about 0.1 wt % to about 7.0 wt %. In some embodiments, 2-(3,4,5-trimethoxyphenyl)acetamide is between about 0.1 wt % to about 6.0 wt %. In some embodiments, 2-(3,4,5-trimethoxyphenyl)acetamide is between about 0.1 wt % to about 5.0 wt %. In some embodiments, 2-(3,4,5-trimethoxyphenyl)acetamide is between 0.1 wt % to 7.0 wt %. In some embodiments, 2-(3,4,5-trimethoxyphenyl)acetamide is between 0.1 wt % to 6.0 wt %. In some embodiments, 2-(3,4,5-trimethoxyphenyl)acetamide is between 0.1 wt % to about 5.0 wt %.

In another aspect, the present disclosure provides a composition comprising a compound represented by Formula (III):

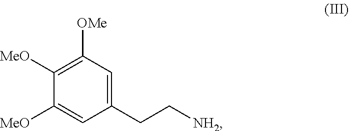

wherein, the composition is characterized by one or more additional characteristics selected from: from: (i) between 0.1 wt % to 8.0 wt % 2-(3,4,5-trimethoxyphenyl)acetamide; (ii) between 0.1 wt % to 5 wt % dimeric amine; (iii) between 0.1 wt % to 5 wt % n-butanol; and (iv) between 0.1 wt % to 6% toluene. In some embodiments, the composition is characterized by two or more characteristics selected from i to iv. In some embodiments, the composition is characterized by at least two or more characteristics selected from i to iv. In some embodiments, the composition is characterized by at least three or more characteristics selected from i to iv. In some embodiments, the composition is characterized by at least four or more characteristics selected from i to iv. In some embodiments, the composition is characterized by at least five or more characteristics selected from i to iv. In some embodiments, the composition is characterized by at least six characteristics selected from i to iv. In some embodiments, 2-(3,4,5-trimethoxyphenyl)acetamide is represented by

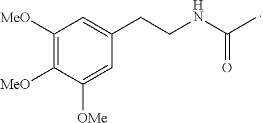 [SMILES: COc1cc(CCNC(C)=O)cc(OC)c1OC]

In some embodiments, 2-(3,4,5-trimethoxyphenyl)acetamide is between 0.1 wt % to 7.0 wt %. In some embodiments, 2-(3,4,5-trimethoxyphenyl)acetamide is between 0.1 wt % to 6.0 wt %. In some embodiments, 2-(3,4,5-trimethoxyphenyl)acetamide is between 0.1 wt % to about 5.0 wt %.

In another aspect, the present disclosure provides a composition comprising a compound represented by (IV):

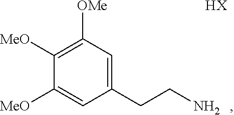

wherein the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between about 0.01 ppm to about 0.2 ppm Al. In some embodiments, the composition is characterized by two or more characteristics selected from i to v. In some embodiments, the composition is characterized by at least two characteristics selected from i to v. In some embodiments, the composition is characterized by at least three characteristics selected from i to v. In some embodiments, the composition is characterized by at least four characteristics selected from i to v. In some embodiments, the composition is characterized by five characteristics selected from i to v.

In some embodiments, the composition comprises a compound represented by (IV):

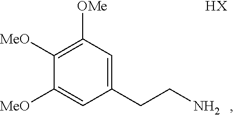

wherein the composition is characterized by one or more additional characteristics selected from: (i) between 0.1 wt % to 2 wt % isopropyl alcohol; (ii) between 0.01 wt % to 0.1 wt % ethanol; (iii) between 0.01 wt % to 0.25 wt % water; (iv) between 0.1 ppm to 1 ppm Li; and (v) between 0.01 ppm to 0.2 ppm Al. In some embodiments, the composition is characterized by two or more characteristics selected from i to v. In some embodiments, the composition is characterized by at least two characteristics selected from i to v. In some embodiments, the composition is characterized by at least three characteristics selected from i to v. In some embodiments, the composition is characterized by at least four characteristics selected from i to v. In some embodiments, the composition is characterized by five characteristics selected from i to v.

In some embodiments, the composition comprises a compound represented by (IV):

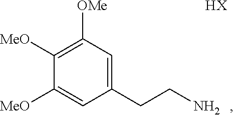

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; and (iii) between about 0.01 wt % to about 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV):

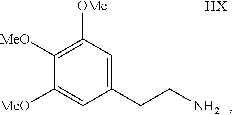

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; (iii) between about 0.01 wt % to about 5 wt % 5-(2-aminoethyl)-2,3-dimethoxyphenol; and (iv) between about 0.1 wt % to about 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV):

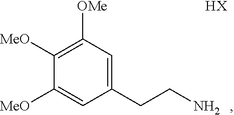

wherein the composition is characterized by one or more additional characteristics selected from: (i) between about 1 ppm to about 50 ppm isopropyl alcohol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; and (vii) between about 0.1 ppm to about 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV):

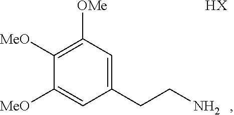

wherein the composition is characterized by one or more additional characteristics selected from: (i) between about 1 ppm to about 50 ppm isopropanol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; (vii) between about 0.1 ppm to about 200 ppm Al; (viii) between about 0.1 wt % to about 8.0 wt % 2-(3,4,5-trimethoxyphenyl)acetamide; (ix) between about 0.01 wt % to about 5 wt % 4-(2-aminoethyl)-2,6-dimethoxyphenol; and (x) between about 0.01 wt % to about 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

In some embodiments, the composition comprises a compound represented by (IV):

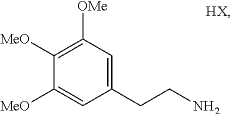 [SMILES: COc1cc(CCN)cc(OC)c1OC]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; and (iii) between 0.01 wt % to 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV):

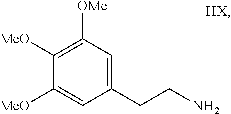 [SMILES: COc1cc(CCN)cc(OC)c1OC]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; (iii) between 0.01 wt % to 5 wt % 5-(2-aminoethyl)-2,3-dimethoxyphenol; and (iv) between 0.1 wt % to 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV):

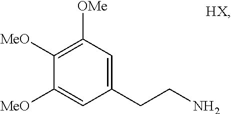 [SMILES: COc1cc(CCN)cc(OC)c1OC]

wherein the composition is characterized by one or more additional characteristics selected from: (i) between 1 ppm to 50 ppm isopropyl alcohol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; and (vii) between 0.1 ppm to 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV):

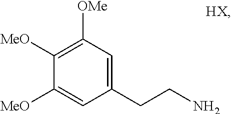 [SMILES: COc1cc(CCN)cc(OC)c1OC]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

In another aspect, the present disclosure provides a composition comprising a compound represented by (IV-a):

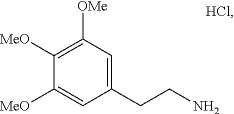 [SMILES: COc1cc(CCN)cc(OC)c1OC]

wherein the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between about 0.01 ppm to about 0.2 ppm Al. In some embodiments, the composition is characterized by two or more characteristics selected from i to v. In some embodiments, the composition is characterized by at least two characteristics selected from i to v. In some embodiments, the composition is characterized by at least three characteristics selected from i to v. In some embodiments, the composition is characterized by at least four characteristics selected from i to v. In some embodiments, the composition is characterized by five characteristics selected from i to v.

In some embodiments, the composition comprises a compound represented by (IV-a):

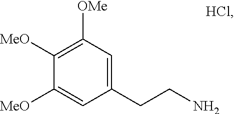 [SMILES: COc1cc(CCN)cc(OC)c1OC]

wherein the composition is characterized by one or more additional characteristics selected from: (i) between 0.1 wt % to 2 wt % isopropyl alcohol; (ii) between 0.01 wt % to 0.1 wt % ethanol; (iii) between 0.01 wt % to 0.25 wt % water; (iv) between 0.1 ppm to 1 ppm Li; and (v) between 0.01 ppm to 0.2 ppm Al. In some embodiments, the composition is characterized by two or more characteristics selected from i to v. In some embodiments, the composition is characterized by at least two characteristics selected from i to v. In some embodiments, the composition is characterized by at least three characteristics selected from i to v. In some embodiments, the composition is characterized by at least four characteristics selected from i to v. In some embodiments, the composition is characterized by five characteristics selected from i to v.

In some embodiments, the composition comprises a compound represented by (IV-a):

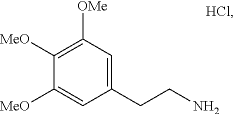 [SMILES: COc1cc(CCN)cc(OC)c1OC]

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; and (iii) between about 0.01 wt % to about 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV-a):

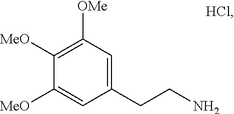 [SMILES: COc1cc(CCN)cc(OC)c1OC]

wherein the composition is characterized by one or more additional characteristics selected from: (i) between about 0.1 wt % to about 8.0 wt % 2-(3,4,5-trimethoxyphenyl)acetamide; (ii) between about 0.01 wt % to about 5 wt % 4-(2-aminoethyl)-2,6-dimethoxyphenol; (iii) between about 0.01 wt % to about 5 wt % 5-(2-aminoethyl)-2,3-dimethoxyphenol; and (iv) between about 0.1 wt % to about 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV-a):

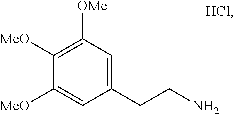 [SMILES: COc1cc(CCN)cc(OC)c1OC]

wherein the composition is characterized by one or more additional characteristics selected from: (i) between about 1 ppm to about 50 ppm isopropyl alcohol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; and (vii) between about 0.1 ppm to about 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV-a):

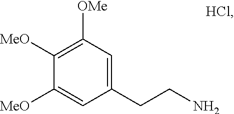 [SMILES: COc1cc(CCN)cc(OC)c1OC]

wherein the composition is characterized by one or more additional characteristics selected from: (i) between about 1 ppm to about 50 ppm isopropanol; (ii) between about 1 ppm to about 50 ppm methanol; (iii) between about 500 ppm to about 1000 ppm ethanol; (iv) between about 0.01 wt % to about 0.25 wt % water; (v) between about 10 wt % to about 20 wt % chloride; (vi) between about 0.1 ppm to about 10 ppm Li; (vii) between about 0.1 ppm to about 200 ppm Al; (viii) between about 0.1 wt % to about 8.0 wt % 2-(3,4,5-trimethoxyphenyl)acetamide; (ix) between about 0.01 wt % to about 5 wt % 4-(2-aminoethyl)-2,6-dimethoxyphenol; and (x) between about 0.01 wt % to about 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

In some embodiments, the composition comprises a compound represented by (IV-a):

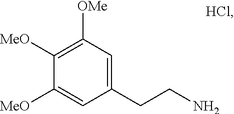 [SMILES: COc1cc(CCN)cc(OC)c1OC]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; and (iii) between 0.01 wt % to 5 wt % 5-(2-aminoethyl)-2,3-dimethoxyphenol. In some embodiments, the composition is characterized by two or more characteristics selected from i to iii. In some embodiments, the composition is characterized by three characteristics selected from i to iii.

In some embodiments, the composition comprises a compound represented by (IV-a):

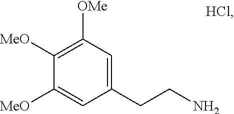 [SMILES: COc1cc(CCN)cc(OC)c1OC]

wherein the composition is characterized by one or more additional characteristics selected from: (i) between 0.1 wt % to 8.0 wt % 2-(3,4,5-trimethoxyphenyl)acetamide; (ii) between 0.01 wt % to 5 wt % 4-(2-aminoethyl)-2,6-dimethoxyphenol; (iii) between 0.01 wt % to 5 wt % 5-(2-aminoethyl)-2,3-dimethoxyphenol; and (iv) between 0.1 wt % to 5% isopropanol. In some embodiments, the composition is characterized by two or more characteristics selected from i to iv. In some embodiments, the composition is characterized by three or more characteristics selected from i to iv. In some embodiments, the composition is characterized by four characteristics selected from i to iv.

In some embodiments, the composition comprises a compound represented by (IV-a):

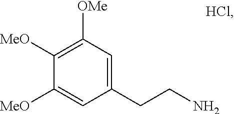 [SMILES: COc1cc(CCN)cc(OC)c1OC]

wherein the composition is characterized by one or more additional characteristics selected from: (i) between 1 ppm to 50 ppm isopropyl alcohol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; and (vii) between 0.1 ppm to 200 ppm Al. In some embodiments, the composition is characterized by two or more characteristics selected from i to vii. In some embodiments, the composition is characterized by three or more characteristics selected from i to vii. In some embodiments, the composition is characterized by four or more characteristics selected from i to vii. In some embodiments, the composition is characterized by five or more characteristics selected from i to vii. In some embodiments, the composition is characterized by six or more characteristics selected from i to vii. In some embodiments, the composition is characterized by seven characteristics selected from i to vii.

In some embodiments, the composition comprises a compound represented by (IV-a):

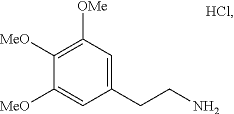 [SMILES: COc1cc(CCN)cc(OC)c1OC]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the composition is characterized by two or more characteristics selected from i to x. In some embodiments, the composition is characterized by three or more characteristics selected from i to x. In some embodiments, the composition is characterized by four or more characteristics selected from i to x. In some embodiments, the composition is characterized by five or more characteristics selected from i to x. In some embodiments, the composition is characterized by six or more characteristics selected from i to x. In some embodiments, the composition is characterized by seven or more characteristics selected from i to x. In some embodiments, the composition is characterized by eight or more characteristics selected from i to x. In some embodiments, the composition is characterized by nine or more characteristics selected from i to x.

### Treatment of Substance Use Disorders Using Pure Forms of Mescaline

In certain aspects, the present disclosure provides methods of treating a substance use disorder with a mescaline or a salt thereof. In some embodiments, the mescaline or salt thereof is a composition of Formula (IV). In some embodiments, the mescaline or salt thereof is a composition of Formula (IV-a). In some embodiments, the mescaline or a salt thereof is synthetic mescaline. In some embodiments, the mescaline or salt thereof is a composition of Formula (IV). In some embodiments, the mescaline or salt thereof is a composition of Formula (IV-a). In some embodiments, the composition of Formula (IV-a) is a composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol.

In some embodiments, the method of treating a substance use disorder comprises administering to a subject in need thereof a composition that comprises a compound represented by (IV):

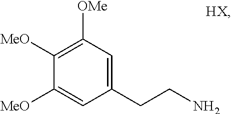 [SMILES: COc1cc(CCN)cc(OC)c1OC]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating a substance use disorder comprises administering to a subject in need thereof a composition that comprises a compound represented by (IV-a):

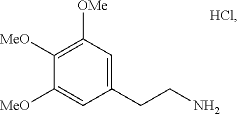 [SMILES: COc1cc(CCN)cc(OC)c1OC ; "HCl,"]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating a substance use disorder comprises administering to a subject in need thereof a composition that comprises a compound represented by (IV-a):

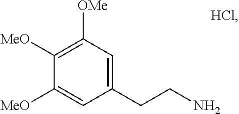 [SMILES: COc1cc(CCN)cc(OC)c1OC ; "HCl,"]

wherein the composition is characterized by two or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating a substance use disorder comprises administering to a subject in need thereof a composition that comprises a compound represented by (IV-a):

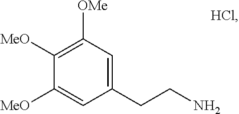 [SMILES: COc1cc(CCN)cc(OC)c1OC ; "HCl,"]

wherein the composition is characterized by three or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the present disclosure provides a method of treating an alcohol use disorder comprising: administering to a subject in need thereof a composition that comprises a compound represented by (IV):

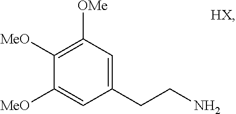 [SMILES: COc1cc(CCN)cc(OC)c1OC ; "HX,"]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating an alcohol use disorder comprises administering to a subject in need thereof a composition that comprises a compound represented by (IV-a):

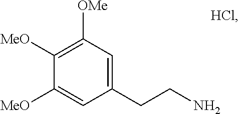 [SMILES: COc1cc(CCN)cc(OC)c1OC ; "HCl,"]

wherein the composition is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating an alcohol use disorder comprises administering to a subject in need thereof a composition that comprises a compound represented by (IV-a):

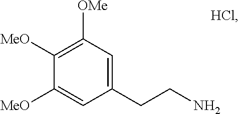 [SMILES: COc1cc(CCN)cc(OC)c1OC ; "HCl,"]

wherein the composition is characterized by two or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

In some embodiments, the method of treating an alcohol use disorder comprises administering to a subject in need thereof a composition that comprises a compound represented by (IV-a):

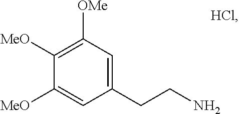 [SMILES: COc1cc(CCN)cc(OC)c1OC ; "HCl,"]

wherein the composition is characterized by three or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol. In some embodiments, the additional characteristic is between 1 ppm to 50 ppm isopropanol. In some embodiments, the additional characteristic is between 1 ppm to 50 ppm methanol. In some embodiments, the additional characteristic is between 500 ppm to 1000 ppm ethanol. In some embodiments, the additional characteristic is between 0.01 wt % to 0.25 wt % water. In some embodiments, the additional characteristic is between 10 wt % to 20 wt % chloride. In some embodiments, the additional characteristic is between 0.1 ppm to 10 ppm Li. In some embodiments, the additional characteristic is between 0.1 ppm to 200 ppm Al. In some embodiments, the additional characteristic is between 0.1 wt % to 8.0 wt % 2-(3,4,5-trimethoxyphenyl)acetamide. In some embodiments, the additional characteristic is between 0.01 wt % to 5 wt % 4-(2-aminoethyl)-2,6-dimethoxyphenol. In some embodiments, the additional characteristic is between 0.01 wt % to 5 wt % 5-(2-aminoethyl)-2,3-dimethoxyphenol.

### (d) Methods of Treating Substance Use Disorders Using Synthetic Mescaline

In certain aspects, the present disclosure provides methods of treating substance use disorders using synthetic mescaline (e.g., mescaline synthesized using one or more synthetic steps). The synthetic mescaline disclosed herein provides clinical advantages over extractions of mescaline from natural sources (e.g., plants or cacti), such as high chemical purity and batch-to-batch consistency, thereby facilitating consistent and reproducible dosing. In some embodiments, the synthetic mescaline disclosed herein is not extracted from plants or cacti containing mescaline. In some embodiments, the synthetic mescaline disclosed herein is produced using multiple synthetic steps. In some embodiments, the synthetic mescaline is substantially free of plant cells or plant tissue. In some embodiments, the synthetic mescaline contains less than 5% of plant cells or plant tissue. In some embodiments, the synthetic mescaline contains less than 1% of plant cells or plant tissue. In some embodiments, the synthetic mescaline is represented by Formula (IV). In some embodiments, the synthetic mescaline is represented by Formula (IV-a). In some embodiments, the method of a method for treating a substance use disorder, comprising: (a) identifying a subject who has a substance use disorder; and (b) orally administering a pharmaceutical composition comprising synthetic mescaline or a salt thereof to the subject and accompanying said administration with a therapy session.

In some embodiments, the subject is identified with a substance use disorder by a medical professional using symptoms or criteria as defined by DSM-5. In some embodiments, the substance use disorder in a subject is diagnosed by a medical professional. In some embodiments, the substance use disorder in a subject is diagnosed as a mild substance use disorder. In some embodiments, the substance use disorder in a subject is diagnosed as a moderate substance use disorder. In some embodiments, the substance use disorder in a subject is diagnosed as a severe substance use disorders. In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalants use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism.

In some embodiments, the synthetic mescaline was produced in accordance with Good Manufacturing Practices (GMP) guidelines. In some embodiments, the synthetic mescaline was produced in a GMP facility.

In some embodiments, the synthetic mescaline comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the synthetic mescaline comprises less than 5% impurities. In some embodiments, the synthetic mescaline comprises less than 1% impurities. In some embodiments, the synthetic mescaline comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the synthetic mescaline comprises an impurity selected from plant cells, plant tissue, and alkaloids. In some embodiments, the synthetic mescaline impurity is an alkaloid. In some embodiments, the alkaloid is a naturally occurring alkaloid. In some embodiments the alkaloid is selected from N-methylmescaline, N-acetylmescaline, hordenine, pellotine, anhalonine, lobivine, tyramine, N-methyltyramine, anhalidine, anhalonidine, O-methyl-anhalonidine, lophophine, lophophorine, anhalinine, anhalamine, homopiperonylamine, and a combination thereof. In some embodiments, the alkaloid is represented by

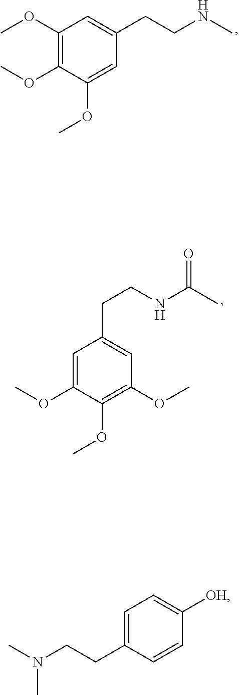

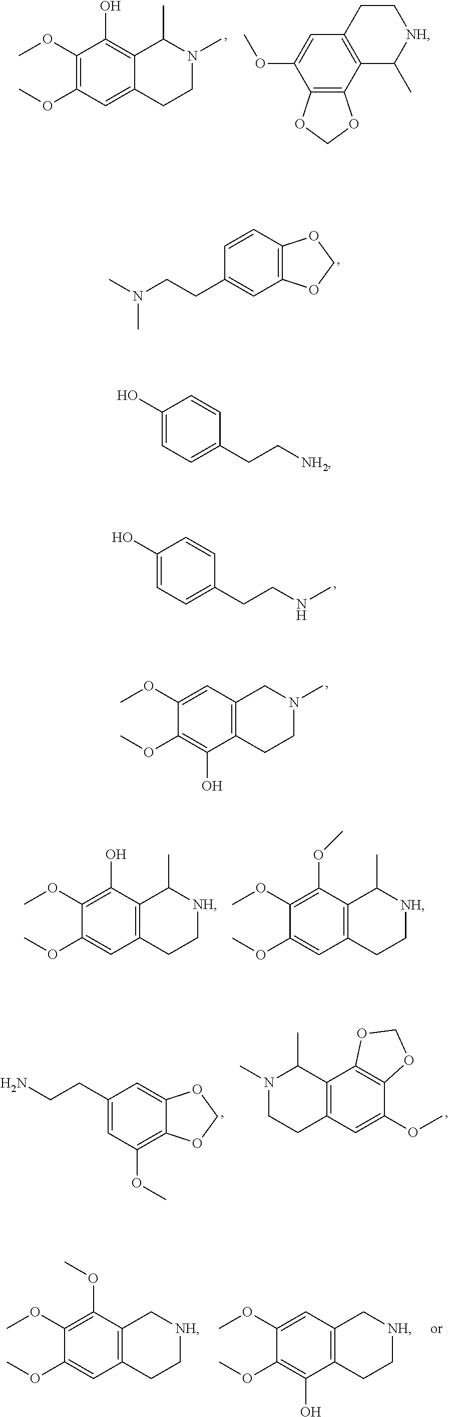

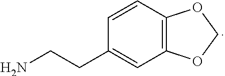 [SMILES: NCCc1ccc2c(c1)OCO2]

In some embodiments, the synthetic mescaline comprises an impurity selected from plant cells and plant tissue. In some embodiments, the impurity is selected from plant cells. In some embodiments, the impurity is selected from plant tissue. In some embodiments, the plant cells or plant tissue are derived from a cactaceae plant, a fabaceae plant, or a combination thereof. In some embodiments, the plant cells or plant tissue are derived from a plant is selected from Lophophora williamsii, Trichocereus (Echinopsis) pachanoi, Echinopsis peruviana, Trichocereus bridgesii, Pereskia aculeata, Acacia berlandieri, Pelecyphora aselliformis, and a combination thereof.

In some embodiments, the synthetic mescaline is represented by Formula (IV):

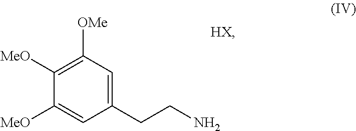 [SMILES: COc1cc(CCN)cc(OC)c1OC ; "HX,"]

wherein HX is an acid and X represents the conjugate base of the acid.

In some embodiments, the pharmaceutical composition comprises an acid addition salt of mescaline selected from an HCl addition salt and a sulfuric acid addition salt.

In some embodiments, the pharmaceutical composition comprises mescaline HCl.

In some embodiments, the synthetic mescaline is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol.

In some embodiments, the synthetic mescaline is characterized by two or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol.

In some embodiments, the synthetic mescaline is characterized by three or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol.

In some embodiments, the pharmaceutical composition comprises from 50-2000 mg of synthetic mescaline or a salt thereof. In some embodiments, the pharmaceutical composition comprises from 50-1500 mg of synthetic mescaline or a salt thereof. In some embodiments, the pharmaceutical composition comprises from 50-1000 mg of synthetic mescaline or a salt thereof. In some embodiments, the pharmaceutical composition comprises from 50-900 mg of synthetic mescaline or a salt thereof. In some embodiments, the pharmaceutical composition comprises from 50-800 mg of synthetic mescaline or a salt thereof. In some embodiments, the pharmaceutical composition comprises from 50-700 mg of synthetic mescaline or a salt thereof. In some embodiments, the pharmaceutical composition comprises from 50-600 mg of synthetic mescaline or a salt thereof. In some embodiments, the pharmaceutical composition comprises from 50-500 mg of synthetic mescaline or a salt thereof. In some embodiments, the pharmaceutical composition comprises from 100-400 mg of synthetic mescaline or a salt thereof. In some embodiments, the pharmaceutical composition comprises from 50-300 mg of synthetic mescaline or a salt thereof. In some embodiments, the pharmaceutical composition comprises from 50-200 mg of synthetic mescaline or a salt thereof. In some embodiments, the pharmaceutical composition comprises from 200-2000 mg of synthetic mescaline or a salt thereof. In some embodiments, the pharmaceutical composition comprises from 300-2000 mg of synthetic mescaline or a salt thereof. In some embodiments, the pharmaceutical composition comprises from 400-2000 mg of synthetic mescaline or a salt thereof. In some embodiments, the pharmaceutical composition comprises from 500-2000 mg of synthetic mescaline or a salt thereof. In some embodiments, the pharmaceutical composition comprises from 600-2000 mg of synthetic mescaline or a salt thereof. In some embodiments, the pharmaceutical composition comprises from 700-2000 mg of synthetic mescaline or a salt thereof. In some embodiments, the pharmaceutical composition comprises from 800-2000 mg of synthetic mescaline or a salt thereof. In some embodiments, the pharmaceutical composition comprises from 900-2000 mg of synthetic mescaline or a salt thereof. In some embodiments, the pharmaceutical composition comprises from 1000-2000 mg of synthetic mescaline or a salt thereof. In some embodiments, the pharmaceutical composition comprises from 1500-2000 mg of synthetic mescaline or a salt thereof. In some embodiments, the pharmaceutical composition comprises about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, or about 2000 mg of synthetic mescaline or a salt thereof. In some embodiments, the pharmaceutical composition comprises at least 50 mg, 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1500 mg, or at least 2000 mg of synthetic mescaline or a salt thereof. In some embodiments, the pharmaceutical composition at most 50 mg, at most 100 mg, at most 150 mg, at most 200 mg, at most 250 mg, at most 300 mg, at most 350 mg, at most 400 mg, at most 450 mg, at most 500 mg, at most 550 mg, at most 600 mg, at most 650 mg, at most 700 mg, at most 750 mg, at most 800 mg, at most 850 mg, at most 900 mg, at most 950 mg, at most 1000 mg, at most 1500 mg, or at most 2000 mg of synthetic mescaline or a salt thereof. In some embodiments, the pharmaceutical composition comprises from 50-2000 mg of synthetic mescaline or a salt thereof. In some embodiments, the pharmaceutical composition comprises from 50-700 mg of synthetic mescaline or a salt thereof.

In some embodiments, the pharmaceutical composition is in a tablet or a capsule. In some embodiments, the pharmaceutical composition is in a tablet. In some embodiments, the pharmaceutical composition is in a capsule. In some embodiments, the pharmaceutical composition is in a tablet or a capsule, and comprises less than 5% impurities. In some embodiments, the pharmaceutical composition is in a tablet or a capsule, and 80% or more of the pharmaceutical composition comprises the synthetic mescaline or salt thereof.

In some embodiments, 80% or more of the pharmaceutical composition comprises the synthetic mescaline or salt thereof. In some embodiments, 85% or more of the pharmaceutical composition comprises the synthetic mescaline or salt thereof. In some embodiments, 90% or more of the pharmaceutical composition comprises the synthetic mescaline or salt thereof. In some embodiments, 95% or more of the pharmaceutical composition comprises the synthetic mescaline or salt thereof. In some embodiments, 99% or more of the pharmaceutical composition comprises the synthetic mescaline or salt thereof.

In some embodiments, the identifying of step (a) comprises screening a subject before step (b). In some embodiments, the screening comprises selecting a subject that has naïve psychedelic exposure or limited psychedelic exposure. In another embodiment, the naïve psychedelic exposure means that a subject had not ever consumed a psychedelic substance prior to step (a). In another embodiment, the limited psychedelic exposure means that a subject has consumed psychedelic substances less than 10 times in their lifetime, prior to step (a).

In some embodiments, the screening further comprises a reviewing step. In some embodiments, the screening comprises reviewing family psychiatric history for a relevant psychiatric disorder in first-degree relatives or second-degree relatives. In some embodiments, a subject may be excluded from step (b) if the subject's first-degree relative has a psychiatric disorder. In some embodiments, a subject may be excluded from step (b) if the subject's second-degree relative has a psychiatric disorder. In some embodiments, the screening comprises reviewing the subject's previous use of psychedelics. In some embodiments, the psychedelics are selected from psilocybin, LSD, DMT, ayahuasca, mescaline, ibogaine, 2C-drugs (such as 2CB, 2CI and 2CE), THC, and ketamine. In some embodiments, the screening comprises reviewing the subject's previous use of psychedelics selected from psilocybin, LSD, DMT, ayahuasca, mescaline, ibogaine, 2CB, 2CI, 2CE, THC, and ketamine. In some embodiments, a subject may be excluded from step (b) if they had previous use of psychedelics. In some embodiments, the screening comprises reviewing the subject for a psychiatric adverse effect associated from the previous use of psychedelics. In some embodiments, the psychiatric adverse effect is selected from anxiety, sense of loss of control, paranoid ideation, paranoid behavior, hallucinatory behavior, euphoria, and suicidal ideation. In some embodiments, the screening comprises reviewing the subject for a psychiatric adverse effect selected from anxiety, sense of loss of control, paranoid ideation, paranoid behavior, hallucinatory behavior, euphoria, and suicidal ideation associated with the previous use of psychedelics. In some embodiments, a subject may be excluded from step (b) if they had psychiatric adverse effects associated with the previous use of psychedelics. In some embodiments, the screening comprises reviewing the subject's persistent psychological effects. In some embodiments, the persistent psychological effects are selected from anxiety, depressed mood, paranoid ideation, hallucinations, flash-backs and recurrent flash-backs. In some embodiments, the screening comprises reviewing the subject for a history of suicidal ideation, suicidal behavior, or a history of suicidal attempts. In some embodiments, a subject may be excluded from step (b) if they had persistent psychological effects. In some embodiments, a subject may be excluded from step (b) if they have a history of suicidal ideation, suicidal behavior, or a history of suicidal attempts. In some embodiments, the method further comprises obtaining informed written consent from the subject prior to step (b).

In some embodiments, the method further comprises a preparatory therapy session prior to step (b). In some embodiments, the method further comprises a preparatory therapy session prior to step (b) to prepare the subject for the possible psychological and physiological effects of the administering of step (b). In some embodiments, the method further comprises a preparatory therapy session prior to step (b) to prepare the subject for the possible psychological effects of the administering of step (b). In some embodiments, the method further comprises a preparatory therapy session prior to step (b) to prepare the subject for the possible physiological effects of the administering of step (b). In some embodiments, the preparatory therapy session is provided by a licensed healthcare professional. In some embodiments, the preparatory therapy session comprises a set of instructions listing the psychological and physiological effects of the administering of step (b). In some embodiments, the preparatory therapy session comprises a provision of informed written consent by the subject.

In some embodiments, the subject is instructed to refrain from eating food for 1 to 24 hours prior to the administration of step (b). In some embodiments, the subject is instructed to refrain from eating food for 2 to 24 hours prior to the administration of step (b). In some embodiments, the subject is instructed to refrain from eating food for 3 to 24 hours prior to the administration of step (b). In some embodiments, the subject is instructed to refrain from eating food for 4 to 24 hours prior to the administration of step (b). In some embodiments, the subject is instructed to refrain from eating food for 5 to 24 hours prior to the administration of step (b). In some embodiments, the subject is instructed to refrain from eating food for 6 to 24 hours prior to the administration of step (b). In some embodiments, the subject is instructed to refrain from eating food for 7 to 24 hours prior to the administration of step (b). In some embodiments, the subject is instructed to refrain from eating food for 8 to 24 hours prior to the administration of step (b). In some embodiments, the subject is instructed to refrain from eating food for 9 to 24 hours prior to the administration of step (b). In some embodiments, the subject is instructed to refrain from eating food for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the administration of step (b).

In some embodiments, the subject is instructed to refrain from eating food for at least 1 to 10 hours prior to the administration of step (b). In some embodiments, the subject is instructed to refrain from eating food for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours prior to the administration of step (b). In some embodiments, the subject is instructed to refrain from eating food for at least 10 hours prior to the administration of step (b). In some embodiments, the subject is instructed to refrain from eating food for at least 12 hours prior to the administration of step (b). In some embodiments, the subject is instructed to refrain from eating food for at least 24 hours prior to the administration of step (b).

In some embodiments, step (b) further comprises physiological monitoring of the subject. In some embodiments, the physiological monitoring of the subject comprises monitoring of a physiological parameter selected from supine blood pressure, pulse rate, respiratory rate, and body temperature. In some embodiments, the physiological parameter is supine blood pressure. In some embodiments, the physiological parameter is the pulse rate. In some embodiments, the physiological parameter is the respiratory rate. In some embodiments, the physiological parameter is the body temperature.

In some embodiments, step (b) further comprises psychological monitoring of the subject. In some embodiments, the psychological monitoring of the subject comprises monitoring for negative psychological reactions to treatment. In some embodiments, the psychological monitoring of the subject comprises monitoring changes in mood in the subject. In some embodiments, the changes in mood evaluated using questionaries. In some embodiments, the changes in mood evaluated using questionaries sale administered by the subject. In some embodiments, the changes in mood evaluated using questionnaire selected from a Psychological Insight Questionnaire (PIQ), Psychological Insight Scale (PIS), Challenging Experience Questionnaire (CEQ), Emotional Breakthrough Inventory (EBI), and Persisting Effects Questionnaire (PEQ).

In some embodiments, step (b) is monitored by a licensed healthcare professional. In some embodiments, the licensed healthcare professional is selected from a medical doctor, a psychiatrist, a clinical psychologist, a counseling psychologist, a nurse practitioner, a physician assistant, a registered nurse, a clinical scientist, an addiction specialist, a substance abuse counselor, and a therapist. In some embodiments, step (b) is monitored by a medical doctor. In some embodiments, step (b) is monitored by a psychiatrist. In some embodiments, step (b) is monitored by a clinical psychologist. In some embodiments, step (b) is monitored by a counseling psychologist. In some embodiments, step (b) is monitored by a nurse practitioner. In some embodiments, step (b) is monitored by a physician assistant. In some embodiments, step (b) is monitored by a registered nurse. In some embodiments, step (b) is monitored by a clinical scientist. In some embodiments, step (b) is monitored by an addiction specialist. In some embodiments, step (b) is monitored by a substance abuse counselor. In some embodiments, step (b) is monitored by a therapist.

In some embodiments, the subject monitored by a licensed healthcare professional for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours after step (b). In some embodiments, the subject monitored by a licensed healthcare professional for up to 24 hours after step (b). In some embodiments, the subject monitored by a licensed healthcare professional for up to 16 hours after step (b). In some embodiments, the subject monitored by a licensed healthcare professional for up to 12 hours after step (b). In some embodiments, the subject monitored by a licensed healthcare professional for up to 6 hours after step (b). In some embodiments, the subject monitored by a licensed healthcare professional for up to 3 hours after step (b). In some embodiments, the subject monitored by a licensed healthcare professional for up to 2 hours after step (b).

In some embodiments, the subject is domiciled in a center for step (b). In some embodiments, the center is a clinic. In some embodiments, the center is an addiction treatment center. In some embodiments, the subject is domiciled at the center for at least 30 days before step (b). In some embodiments, the subject is domiciled at the center for at least 1 day, at least 2 days, at least 3 days, at least 4 days, at least 5 days, at least 6 days, at least 7 days, at least 14 days, at least 21 days, at least 28 days, or at least 30 days prior to step (b). In some embodiments, the subject is domiciled at the center for 30 days before step (b). In some embodiments, the subject is domiciled at the center for 1 day, 2 days, 3 days, 4 days, 5 days, 6 days, 7 days, 14 days, 21 days, 28 days, or 30 days prior to step (b). In some embodiments, the subject is domiciled for at least 30 days after step (b). In some embodiments, the subject is domiciled at the center for at least 1 day, at least 2 days, at least 3 days, at least 4 days, at least 5 days, at least 6 days, at least 7 days, at least 14 days, at least 21 days, at least 28 days, or at least 30 days after step (b). In some embodiments, the subject is domiciled at the center for 30 days before step (b). In some embodiments, the subject is domiciled at the center for 1 day, 2 days, 3 days, 4 days, 5 days, 6 days, 7 days, 14 days, 21 days, 28 days, or 30 days after step (b). In some embodiments, the subject is located outside of a center for step (b).

In some embodiments, the physiological monitoring of the subject comprises monitoring of a plasma biomarker selected from brain-derived neurotrophic factor (BDNF), hypothalamic-pituitary-adrenal (HPA)-axis endocrine measures, and neuroendocrine measures. In some embodiments, the plasma biomarker is a brain-derived neurotrophic factor (BDNF). In some embodiments, the plasma biomarker is a hypothalamic-pituitary-adrenal (HPA)-axis endocrine measures. In some embodiments, the plasma biomarker is a neuroendocrine measures. In some embodiments, a plasma biomarker analysis is conducted before the dosing and after the dosing. In some embodiments, a plasma biomarker analysis is conducted before the dosing or after the dosing. In some embodiments, a plasma biomarker analysis is conducted before the dosing. In some embodiments, a plasma biomarker analysis is conducted after the dosing. In some embodiments, the plasma biomarker is drawn at 0.5 hour, 1 hour, 1.5 hours, 2 hours, 2.5 hours, 3 hours, 4 hours, 6 hours, 8 hours, 12 hours, 24 hours, or 48 hours before the dosing. In some embodiments, the plasma biomarker is drawn at 0.5 hour, 1 hour, 1.5 hours, 2 hours, 2.5 hours, 3 hours, 4 hours, 6 hours, 8 hours, 12 hours, 24 hours, or 48 hours after the dosing. In some embodiments, additional plasma biomarker analysis may be conducted if the subject has an adverse effect.

In some embodiments, the method further comprises a follow-up consultation after step (b). In some embodiments, the follow-up consultation may be conducted by telephone or as a video call visit or as in-clinic visit. In some embodiments, the follow-up visit is conducted by a licensed healthcare professional. In some embodiments, the follow-up consultation comprises psychosocial care or an evaluation. In some embodiments, the follow-up consultation comprises psychosocial care. In some embodiments, the psychosocial care is conducted by a licensed healthcare professional. In some embodiments, the follow-up consultation comprises an evaluation. In some embodiments, the evaluation is conducted by a licensed healthcare professional. In some embodiments, the evaluation comprises a questionnaire, a post-experience evaluation, a breakthrough evaluation, or an additional therapy evaluation. In some embodiments, the evaluation is self-administered by the subject. In some embodiments, the evaluation comprises a questionnaire. In some embodiments, the questionnaire is self-administered by the subject. In some embodiments, the evaluation is selected from a Psychological Insight Questionnaire (PIQ), Psychological Insight Scale (PIS), Challenging Experience Questionnaire (CEQ), Emotional Breakthrough Inventory (EBI), Persisting Effects Questionnaire (PEQ), hallucinogenic Rating Scale (HRS), Mystical Experience Questionnaire 30-item (MEQ-30), 5-Dimensional Altered States of Consciousness Rating Scale (5D-ASC), and Warwick-Edinburgh Mental Well-Being Scale (WEMWBS).

In some embodiments, the follow-up consultation is up to 1 day, 2 days, 3 days, 4 days, 5 days, 6 days, 7 days, 14 days, 21 days, 28 days or 30 days after step (b). In some embodiments, the follow-up consultation is between 1 to 7 days after step (b). In some embodiments, the follow-up consultation is between 1 to 6 days after step (b). In some embodiments, the follow-up consultation is between 1 to 5 days after step (b). In some embodiments, the follow-up consultation is between 1 to 4 days after step (b). In some embodiments, the follow-up consultation is between 1 to 3 days after step (b). In some embodiments, the follow-up consultation is 1 day after step (b). In some embodiments, the follow-up consultation is 2 days after step (b). In some embodiments, the follow-up consultation is 3 days after step (b). In some embodiments, the follow-up consultation is 4 days after step (b). In some embodiments, the follow-up consultation is 5 days after step (b). In some embodiments, the follow-up consultation is 6 days after step (b). In some embodiments, the follow-up consultation is 7 days after step (b). In some embodiments, the follow-up consultation is conducted by a licensed healthcare professional.

In some embodiments, the subject has refrained from using a psychedelic drug for 6 months, 5 months, 4 months, 3 months, 2 months, or 1 month before step (b). In some embodiments, the subject has refrained from using a psychedelic drug for 6 months before step (b). In some embodiments, the subject has refrained from using a psychedelic drug for 5 months before step (b). In some embodiments, the subject has refrained from using a psychedelic drug for 4 months before step (b). In some embodiments, the subject has refrained from using a psychedelic drug for 3 months before step (b). In some embodiments, the subject has refrained from using a psychedelic drug for 2 months before step (b). In some embodiments, the subject has refrained from using a psychedelic drug for 1 month before step (b). In some embodiments, the psychedelic drug is selected from mescaline, LSD, psilocybin, and DMT. In some embodiments, the psychedelic drug is mescaline. In some embodiments, the psychedelic drug is LSD. In some embodiments, the psychedelic drug is psilocybin. In some embodiments, the psychedelic drug is DMT.

In some embodiments, administering said treatment attenuates substance intake by said subject by about 20% or more as compared with the amount of substance intake before said treatment. In some embodiments, the administering said treatment attenuates substance intake by said subject, by about 30%, about 40%, about 50%, about 60%, about 70%, about 80%, about 90%, or about 95% or more as compared with the amount of substance intake before said treatment. In some embodiments, the administering said treatment attenuates substance intake by said subject, by at least 20%, at least 30%, at least 40%, at least 50%, at least 60%, at least 70%, at least 80%, at least 90%, or at least 95% as compared with the amount of substance intake before said treatment.

In some embodiments, for step (a), said subject has reduced substance consumption for at least 1, 2, 3, 4, 5, 6, or 7 days prior to step (b). In some embodiments, for step (a), said subject has reduced substance consumption for at least 3, 4, 5, 6, or 7 days prior to step (b). In some embodiments, for step (a), said subject has reduced substance consumption for more than 1, 2, 3, 4, 5, 6, or 7 days prior to step (b). In some embodiments, for step (a), said subject has reduced substance consumption for more than 3, 4, 5, 6, or 7 days prior to step (b). In some embodiments, for step (a), said subject has reduced substance consumption for at least 2 days prior to step (b). In some embodiments, for step (a), said subject has reduced substance consumption for at least 3 days prior to step (b). In some embodiments, for step (a), said subject has reduced substance consumption for at least 4 days prior to step (b). In some embodiments, for step (a), said subject has reduced substance consumption for at least 5 days prior to step (b). In some embodiments, for step (a), said subject has reduced substance consumption for at least 6 days prior to step (b). In some embodiments, for step (a), said subject has reduced substance consumption for at least 7 days prior to step (b). In some embodiments, for step (a), said subject has reduced substance consumption for more than 2 days prior to step (b). In some embodiments, for step (a), said subject has reduced substance consumption for more than 3 days prior to step (b). In some embodiments, for step (a), said subject has reduced substance consumption for more than 4 days prior to step (b). In some embodiments, for step (a), said subject has reduced substance consumption for more than 5 days prior to step (b). In some embodiments, for step (a), said subject has reduced substance consumption for more than 6 days prior to step (b). In some embodiments, for step (a), said subject has reduced substance consumption for more than 7 days prior to step (b).

In some embodiments, the subject has reduced substance consumption for 1-12 days. In some embodiments, the subject has reduced substance consumption for 2-12 days. In some embodiments, the subject has reduced substance consumption for 3-12 days. In some embodiments, the subject has reduced substance consumption for 4-12 days. In some embodiments, the subject has reduced substance consumption for 5-12 days. In some embodiments, the subject has reduced substance consumption for 5-11 days. In some embodiments, the subject has reduced substance consumption for 5-10 days. In some embodiments, the subject has reduced substance consumption for 5-9 days. In some embodiments, the subject has reduced substance consumption for 5-8 days. In some embodiments, the subject has reduced substance consumption for 5-7 days. In some embodiments, the subject has reduced substance consumption for 5-6 days. In some embodiments, the subject has reduced substance consumption for 6-12 days. In some embodiments, the subject has reduced substance consumption for 7-12 days. In some embodiments, the subject has reduced substance consumption for 8-12 days. In some embodiments, the subject has reduced substance consumption for 9-12 days. In some embodiments, the subject has reduced substance consumption for 10-12 days. In some embodiments, the subject has reduced substance consumption for 11-12 days.

In some embodiments, the method further comprises a mescaline treatment holiday of from 2 weeks to 12 months. In some embodiments, the mescaline treatment holiday is from 2 weeks to 11 months. In some embodiments, the mescaline treatment holiday is from 2 weeks to 10 months. In some embodiments, the mescaline treatment holiday is from 2 weeks to 9 months. In some embodiments, the mescaline treatment holiday is from 2 weeks to 8 months. In some embodiments, the mescaline treatment holiday is from 2 weeks to 7 months. In some embodiments, the mescaline treatment holiday is from 2 weeks to 6 months. In some embodiments, the mescaline treatment holiday is from 2 weeks to 5 months. In some embodiments, the mescaline treatment holiday is from 2 weeks to 4 months. In some embodiments, the mescaline treatment holiday is from 2 weeks to 3 months. In some embodiments, the mescaline treatment holiday is from 2 weeks to 2 months. In some embodiments, the mescaline treatment holiday is from 2 weeks to 1 months. In some embodiments, the mescaline treatment holiday is from 2 weeks to 4 weeks. In some embodiments, the mescaline treatment holiday is from 2 weeks to 3 weeks. In some embodiments, the mescaline treatment holiday is from 1 month, 2 months, 3 months, 4, month, 5 months, 6 months, 7 month, 8 months, 9 months, 10 months, 11 months, or 12 months. In some embodiments, the mescaline treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the therapy session comprises a practitioner and a plurality of subjects, wherein the practitioner abstains from the oral administration of the pharmaceutical composition. In some embodiments, the practitioner is a licensed healthcare professional as disclosed herein. In some embodiments, the therapy session comprises the practitioner and at least one subject. In some embodiments, the therapy session is a group session with the plurality of subjects. In some embodiments, the group session comprises the practitioner and at least 2 subjects. In some embodiments, the therapy session comprises psychosocial therapy.

### (e) Pharmaceutical Formulations

In certain aspects, the present disclosure provides a pharmaceutical composition comprising at least one pharmaceutically acceptable excipient and a compound or salt of Formula (IV). In certain aspects, the present disclosure provides a pharmaceutical composition comprising at least one pharmaceutically acceptable excipient and mescaline or a salt thereof. In some embodiments, the pharmaceutical composition is formulated into a pharmaceutical formulation. Pharmaceutical formulations may be provided in any suitable form, which may depend on the route of administration. In some embodiments, the pharmaceutical composition disclosed herein can be formulated in dosage form for administration to a subject.

In some embodiments, the pharmaceutical composition is formulated for oral, sublingual intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In parenteral administration characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the dosage form is formulated for oral administration, e.g., oral dosage form. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form. Oral dosage forms are either solid, gel or liquid. The solid dosage forms are tablets, capsules, granules, and bulk powders. Types of oral tablets include compressed, chewable lozenges and tablets which may be enteric coated, sugar coated, or film coated. Capsules may be hard or soft gelatin capsules, while granules and powders may be provided in non-effervescent or effervescent form with the combination of other ingredients known to those skilled in the art. In some embodiments, the pharmaceutical composition disclosed herein can be formulated for oral administration. In some embodiments, the pharmaceutical composition disclosed herein can be formulated for sublingual administration. In some embodiments, the pharmaceutical composition of Formula (IV) or a salt thereof, can be formulated for oral administration. In some embodiments, the pharmaceutical composition Formula (IV) or a salt thereof, can be formulated for sublingual administration.

In some embodiments, the disclosure provides a pharmaceutical composition for oral administration comprising mescaline or a salt thereof and a pharmaceutical excipient suitable for oral administration. In some embodiments, the disclosure provides a pharmaceutical composition for oral administration comprising a compound or salt of Formula (IV) and a pharmaceutical excipient suitable for oral administration. The composition may be in the form of a solid, liquid, gel, semi-liquid, or semi-solid. Pharmaceutical compositions of the disclosure suitable for oral administration can be presented as discrete dosage forms, such as hard or soft capsules, cachets, troches, lozenges, or tablets, or liquids or aerosol sprays. The compounds are, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, transdermal administration and oral inhalation via nebulizers, pressurized metered dose inhalers and dry powder inhalers. In some embodiments, mescaline or a salt thereof may be formulated into compositions using techniques and procedures well known in the art (see, e.g., Ansel, Introduction to Pharmaceutical Dosage Forms, Seventh Edition (1999)).

In certain aspects, the present disclosure provides a pharmaceutical composition comprising at least one pharmaceutically acceptable excipient and a compound or salt of Formula (IV). In some embodiments, the pharmaceutical composition is formulated into a pharmaceutical formulation. Pharmaceutical formulations may be provided in any suitable form, which may depend on the route of administration. In some embodiments, the pharmaceutical composition disclosed herein can be formulated in dosage form for administration to a subject.

In some embodiments, the pharmaceutical composition comprises from 50-2000 mg of a compound or salt of Formula (IV). In some embodiments, the pharmaceutical composition comprises from about 50-1500 mg of mescaline or salt thereof. In some embodiments, the pharmaceutical composition comprises from about 50-1000 mg of mescaline or salt thereof. In some embodiments, the pharmaceutical composition comprises from about 50-900 mg of mescaline or salt thereof. In some embodiments, the pharmaceutical composition comprises from about 50-800 mg of mescaline or salt thereof. In some embodiments, the pharmaceutical composition comprises from about 50-700 mg of mescaline or salt thereof. In some embodiments, the pharmaceutical composition comprises from about 50-600 mg of mescaline or salt thereof. In some embodiments, the pharmaceutical composition comprises from about 100-500 mg of a compound or salt of Formula (IV). In some embodiments, the pharmaceutical composition comprises from about 100-400 mg of a compound or salt of Formula (IV). In some embodiments, the pharmaceutical composition comprises from about 100-300 mg of a compound or salt of Formula (IV). In some embodiments, the pharmaceutical composition comprises from about 100-200 mg of a compound or salt of Formula (IV). In some embodiments, the pharmaceutical composition comprises from about 200-1000 mg of a compound or salt of Formula (IV). In some embodiments, the pharmaceutical composition comprises from about 300-1000 mg of a compound or salt of Formula (IV). In some embodiments, the pharmaceutical composition comprises from about 400-1000 mg of a compound or salt of Formula (IV). In some embodiments, the pharmaceutical composition comprises from about 500-1000 mg of a compound or salt of Formula (IV). In some embodiments, the pharmaceutical composition comprises from about 600-1000 mg of a compound or salt of Formula (IV). In some embodiments, the pharmaceutical composition comprises about 50 mg, about 100 mg, about 200 mg, about 300 mg, about 400 mg, about 500 mg, about 600 mg, about 700 mg, about 800 mg, about 900 mg, about 1000 mg, about 1500 mg, or about 2000 mg of a compound or salt of Formula (IV). In some embodiments, the pharmaceutical composition comprises at least 50 mg, at least 100 mg, at least 200 mg, at least 300 mg, at least 400 mg, at least 500 mg, at least 600 mg, at least 700 mg, at least 800 mg, at least 900 mg, at least 1000 mg, at least 1500 mg, or at least 2000 mg of a compound or salt of Formula (IV). In some embodiments, the pharmaceutical composition comprises at most 50 mg, at most 100 mg, at most 200 mg, at most 300 mg, at most 400 mg, at most 500 mg, at most 600 mg, at most 700 mg, at most 800 mg, at most 900 mg, at most 1000 mg, at most 1500 mg, or at most 2000 mg of a compound or salt of Formula (IV).

In some aspects, the compounds or salts thereof, described herein are useful for the treatment of substance use disorders. In some embodiments, the method comprises administering to a patient suffering from a substance use disorder a composition comprising: a compound represented by (IV)

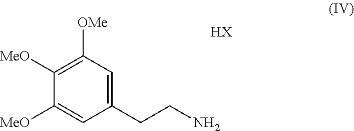

wherein, the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between 0.01 ppm to about 0.2 ppm Al.

In some aspects, the compounds or salts thereof, described herein are useful for the treatment of substance use disorders. In some embodiments, the method comprises administering to a patient suffering from a substance use disorder a composition comprising: a compound represented by (IV-a)

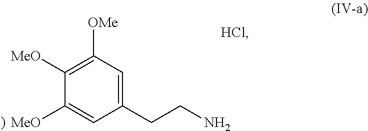

wherein, the composition is characterized by one or more additional characteristics selected from: (i) between about 0.1 wt % to about 2 wt % isopropyl alcohol; (ii) between about 0.01 wt % to about 0.1 wt % ethanol; (iii) between about 0.01 wt % to about 0.25 wt % water; (iv) between about 0.1 ppm to about 1 ppm Li; and (v) between 0.01 ppm to about 0.2 ppm Al.

### EXAMPLES

The following examples are included to further describe some embodiments of the present disclosure and should not be used to limit the scope of the disclosure.

### Example 1: Dosing and Safety Data

Compositions as disclosed herein comprising 50-800 mg of mescaline or salt thereof may be prepared. The compositions may optionally be administered in capsules or tablets comprising 50-800 mg of mescaline or salt thereof. These compositions may then be used to evaluate pharmacokinetic and pharmacodynamic data in vivo. The pharmacology data can then provide effective dosing ranges, toxicity and modes of action, for the treatment of a substance use disorder, for example, AUD.

### Example 2: Treatment of Alcohol Use Disorders

Prior to initial treatment, a subject may undergo preparatory therapy sessions, which involve psychosocial care. The subject may undergo between 1 to 4 preparatory therapy sessions with a medical practitioner. Optionally, during the preparatory therapy sessions, a subject may be evaluated for a reduction in alcohol consumption or a reduction in binge drinking as disclosed herein.

After the preparatory therapy sessions, a subject may be evaluated for a reduction in alcohol consumption or a reduction in binge drinking over a set period of time as disclosed herein. A subject may be given a score based on decrease in heavy drinking days, a reduction in risk alcohol level, or a reduction in binge drinking days. The evaluation may be done periodically and monitored over several days prior to initial treatment. The subject may undergo initial treatment, wherein the subject is administered mescaline or salt thereof, or a pharmaceutical composition comprising mescaline or salt thereof, individually or in a group setting with a medical practitioner. The initial treatment may be administered to a subject based on the subject's score prior to initial treatment. Optionally, the initial treatment may be administered to a subject based on the subject's classification as defined by the National Institute on Alcohol Abuse and Alcoholism (NIAAA) or by the World Health Organization (WHO).

After initial treatment, the subject's progress can be regularly monitored through regular therapy sessions over a period of between two weeks to twelve months. Optionally, the subject may monitor their progress using a self-assessment, instructions for which are provided by a medical practitioner. In some cases, the self-assessment can be conducted outside of the regular therapy session. Additionally, a subject may be administered one or more maintenance sessions after subsequent evaluations by a medical practitioner during regular therapy sessions. The subject may continue regular therapy sessions at the same, or different, interval from before the administration of a maintenance session.

### Example 3: Treatment of Substance Use Disorders

Prior to initial treatment, a subject may optionally undergo preparatory therapy sessions, which involve psychosocial care. The subject may undergo between 1 to 4 preparatory therapy sessions with a medical practitioner. Optionally, during the preparatory therapy sessions, a subject may be diagnosed by a medical professional to evaluate the severity of the subject's substance use disorder using DSM-5 criteria or symptoms.

After the preparatory therapy sessions, a subject may be evaluated for a reduction in substance consumption over a set period of time as disclosed herein. The medical professional's evaluation may be done periodically and monitored over several days prior to initial treatment. The subject may undergo initial treatment, wherein the subject is administered mescaline or salt thereof, or a pharmaceutical composition comprising mescaline or salt thereof, individually or in a group setting with a medical practitioner. The initial treatment may be administered to a subject based on the subject's evaluation prior to initial treatment.

After initial treatment, the subject's progress can be regularly monitored through regular therapy sessions over a period of between two weeks to twelve months. Optionally, the subject may monitor their progress using a self-assessment, instructions for which are provided by a medical practitioner. In some cases, the self-assessment can be conducted outside of the regular therapy session. Additionally, a subject may be administered one or more maintenance sessions after subsequent evaluations by a medical practitioner during regular therapy sessions. The subject may continue regular therapy sessions at the same, or different, interval from before the administration of a maintenance session.

### Example 4—Synthesis of 2-(3,4,5-trimethoxyphenyl)acetamide

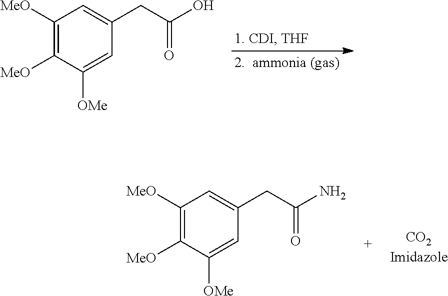 [SMILES: COc1cc(CC(=O)O)cc(OC)c1OC ; COc1cc(CC(N)=O)cc(OC)c1OC]

The use of carbonyldiimidazole (CDI) was subsequently investigated, with DCM as solvent. The formation of the CDI-adduct worked well and subsequent treatment with aqueous ammonia gave a cleaner profile than that obtained from the acid chloride reaction. The use of CDI was subsequently investigated, with DCM as solvent. The formation of the CDI-adduct worked well and subsequent treatment with aqueous ammonia gave a clean profile. However, the isolated yield was low (40%) and it was found that there was significant product in the aqueous layers. The solvent was exchanged with THE in an effort allow for more efficient removal of imidazole during work-up, however after work-up, the level was 22% w/w. The work-up was also challenging, requiring addition of significant amounts of EtOAc and brine to give two layers, with losses of product to the aqueous layer.

The conditions were further modified to address the previous deficiencies. The aqueous/THF conditions were scaled-up to 50 g of acid, using 1.2 eq of CDI in 10 volumes of THF. Both the initial CDI adduct formation and the subsequent treatment with ammonia were successful. A modified work-up procedure gave some improvements, providing 75 g of a white solid that was 57% amide product by NMR (86% active yield), with the remainder being imidazole. This material was recrystallized from THF (5 vols to acid mass), to give 23 g (46% overall yield) of product with a purity of 99.9% by HPLC and >95% by NMR, with imidazole not detected.

A final modification was performed on a 25 gram scale. The formation of the CDI adduct proceeded as expected with 1.2 eq CDI in 10 volumes THF, then the reaction mixture was sparged with ammonia gas for 30 minutes (exotherm to 30° C.). Analysis by HPLC showed complete conversion to 2-(3,4,5-trimethoxyphenyl)acetamide. Unlike previous reactions a thick slurry was formed at this point. A sample of this slurry was filtered and analysis of the solids and filtrates showed product in both, thus the mixture was concentrated and the isolated product purified by THE recrystallization (3 vols to acid starting material).

Recrystallization of this product afforded 14 g (56%) of 2-(3,4,5-trimethoxyphenyl)acetamide, which contained 4 mol % imidazole by NMR. Attempts to purify 2-(3,4,5-trimethoxyphenyl)acetamide using various recrystallization solvents were conducted. However, these were unsuccessful at removing the imidazole impurity.

Base washes were used in lieu of recrystallization of the crude product to afford a higher recovery of 2-(3,4,5-trimethoxyphenyl)acetamide and lower imidazole content. The crude material (5 grams) was dissolved in DCM (10 vols) and washed multiple times with saturated aq. NaHCO₃ (5 vols). This process was repeated with the organic layers combined and dried, which afforded 3.7 grams (79% yield) of product containing 8 mol % imidazole.

In order to assess whether residual imidazole had an effect on the production of mescaline in the next step, a 3.5 g scale reaction was performed using the product that contained 8 mol % imidazole. The reaction worked as expected, with no change in the profile compared to previous batches, demonstrating that up to 8 mol % content imidazole can be tolerated for the subsequent synthetic step.

Table 1 provides the process for the production of 2-(3,4,5-trimethoxyphenyl)acetamide on a 450 gram scale. Final weight of product was 338 grams (75% yield).

TABLE 1
Synthetic Steps for the Synthesis of 2-(3,4,5-trimethylmethoxyphenyl)acetamide

| Step No. | Process |
|---|---|
| 1 | Add 3,4,5-trimethoxyphenyl acetic acid (1.0 g/g, limiting reagent) to the vessel |
| 2 | Add THF (10 mL/g) to the vessel. Turn on the stirrer. Stir for 15 minutes, until the |
| | majority of the solid dissolves. Set jacket to 15-25° C. |
| 3 | Add CDI (0.86 g/g, 1.20 eq) portion wise over at least 40 minutes, allowing it to |
| | dissolve before addition of the next portion. |
| 4 | On completion of addition stir at 15-25° C. for a minimum of 2 hours. |
| 5 | Set up a sparging tube into the vessel and an exhaust pipe into a carboy of water. |
| 6 | Set the jacket to 20° C. |
| 7 | Sparge the reaction with ammonia gas at a rate of ~0.5 L/min for at least 45 minutes |
| | at 20-32° |
| 8 | Cool the mixture back to 15-25° C. |
| 9 | Concentrate the mixture in vacuo |
| 10 | Add the solid back to the reaction vessel. |
| 11 | Add DCM (10 mL/g) to the vessel. Turn on the stirrer. Set the jacket to 15-25° C. |
| 12 | Add saturated aqueous NaHCO₃ (5 mL/g) over 5 min. |
| 13 | Stir the mixture for at least 30 minutes at 15-25° C. |
| 14 | Separate the layers |
| 15 | Recharge the bottom organic layer to the vessel |
| 16 | Add saturated aqueous NaHCO₃ (3 mL/g) over 5 min. |
| 17 | Stir the mixture for at least 20 minutes at 15-25° C. |
| 18 | Separate the layers |
| 19 | Recharge the bottom organic layer to the vessel |
| 20 | Add saturated aqueous NaHCO₃ (3 mL/g) over 5 min. |
| 21 | Stir the mixture for at least 20 minutes at 15-25° C. |
| 22 | Separate the layers |
| 23 | Dry over Na₂SO₄, filter and wash solids with DCM (2 mL/g) |
| 24 | Concentrate the filtrate in vacuo at 40° C. |
| 25 | Dry the solids in vacuo at 40° C. until constant mass is achieved. |

¹H NMR (CDCl₃) provided 98.257 wt % 2-(3,4,5-trimethylmethoxyphenyl)acetamide, 0.556 wt % dichloromethane, and 1.187 wt % imidazole. HPLC provided 96% purity.

### Example 5—Synthesis of Mescaline

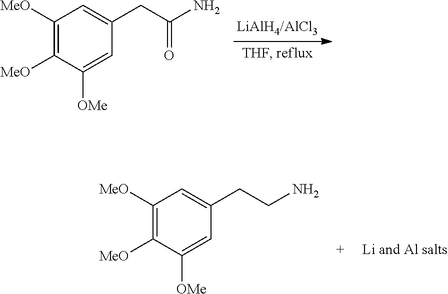 [SMILES: COc1cc(CC(N)=O)cc(OC)c1OC ; COc1cc(CCN)cc(OC)c1OC]

Table 2 provides the process for the production of Mescaline on a 325 gram scale. Final weight of product was 242 grams (79% yield).

TABLE 2
Synthetic Steps for the synthesis of Mescaline

| Step No. | Process |
|---|---|
| 1 | Add THF (2.62 mL/g) to a first vessel. Turn on stirrer. |
| 2 | Add AlCl₃ (0.43 g/g, 0.73 eq) portion wise over 30-60 minutes to the first vessel, keeping |
| | T <30° C., allowing the majority of the solid to dissolve between additions. |
| 3 | Stir the mixture for at least 30 minutes, allowing temperature to return to 15-25° C. |
| 4 | Add LiAlH₄ (2.4M in THF, 4.06 mL/g, 2.2 eq) to the AlCl₃ solution, dropwise over |
| | 30-60 minutes, keeping T <30° C. |
| 5 | Stir the AlCl₃/LiAlH₄ mixture at 15-25° C. for 30 minutes |
| 6 | To a second vessel add 2-(3,4,5-trimethylmethoxyphenyl)acetamide from step 1, (1 g/g, |
| | limiting reagent) |
| 7 | Add THF (5 mL/g). Turn on stirrer |
| 8 | Cool mixture to 5-15° C. |
| 9 | Add AlC13/LiAlH4 mixture from Step 5, dropwise over at least 45 minutes, keeping T <30° C. |
| 10 | On completion of addition heat the mixture to 60-67° C. stir for at least 16 hours |
| 11 | Cool reaction mixture to room temperature (15-25° C.) Divide into 2 equal portions if |
| | carrying out split work-up. |
| 12 | To a clean second vessel. add methanol (0.936 mL/g) |
| 13 | Charge ethyl acetate (17.1 mL/g) |
| 14 | Charge Rochelle salts (11.5 mL/g) (53% aq); 12.2 kg Rochelle salts added to 17440 mL |
| | of water makes 23 L of 53% |
| 15 | Cool the quench mixture to between 5 and 10° C. |
| 16 | Add the reaction mixture into the quench solution (reverse addition) over at least 2 hours |
| | keeping the temp below 30° C. (Either use a dropping funnel for the addition or transfer |
| | the batch into a 20 L flange flask and use N2 push) |
| 17 | Stir the quenched reaction mixture for minimum of 15 minutes. Temp = 15-25° C. |
| 18 | Separate phases. Charge Ethyl acetate (3.95 mL/g) to assist separation. Temp = 15-25° C. |
| 19 | Charge Aq. phase back to the vessel and extract with ethyl acetate (7.36 mL/g) stir |
| | for 15 mins and separate. Charge ethyl acetate (3.94 mL/g) to assist separation. |
| | Temp = 15-25° C. |
| 20 | Charge Aq. phase back to the vessel and extract with Ethyl acetate (7.36 mL/g) stir for 15 |
| | mins and separate. Charge Ethyl acetate (3.94 mL/g) to assist separation. Temp = 15- |
| 21 | Dry combined organic extracts over sodium sulphate |
| 22 | Filter dried organics, and wash with ethyl acetate (1.5 mL/g) |
| 23 | Concentrate combined organics at 40-50° C. under vacuum. Azeotrope with toluene (2 × |
| | 1.5 mL/g). Product is an oil, stored in a duran bottle rinsed in with IPA (1 mL/g) and |
| | purge with N₂. |

NMR of the final material identified 4.2% toluene and 1.6% n-butanol of the product as impurities. HPLC of the final material identified 2-(3,4,5-trimethoxyphenyl)acetamide, dimeric amine (MW 405 g/mol), and additional oligomeric amines as impurities.

### Example 6—Synthesis of Mescaline Hydrochloride

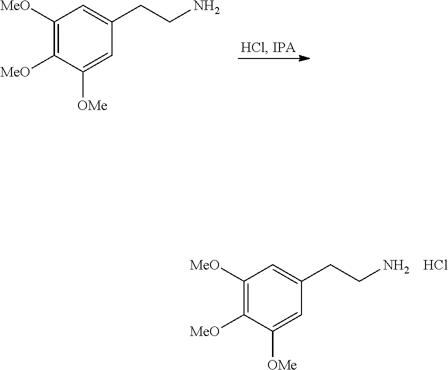

Table 3 provides the process for the production of Mescaline Hydrochloride on a 233 gram scale. Final weight of product was 195 grams (71% yield).

TABLE 3
Synthetic Steps for the Synthesis of Mescaline Hydrochloride

| Step No. | Process |
|---|---|
| 1 | Dissolve Mescaline free base (1.0 g/g active) in IPA (4.5 mL/g) |
| 2 | Polish filter the mescaline free base solution into the vessel at 15-25° C. |
| 3 | Rinse the mescaline container with polish filtered IPA (0.5 mL/g) and use this to |
| | rinse the lines into the vessel. |
| 4 | Turn on the stirrer. Set the jacket to 10° C. |
| 5 | Polish filter the HCl in IPA into an addition funnel by nitrogen push then add to |
| | the vessel dropwise over at least 45-60 minutes, maintaining T <25° C. |
| 6 | On completion of addition cool the mixture to 0-5° C. and stir for 1 hour |
| 7 | Filter the batch |
| 8 | Add polish filtered IPA (0.5 mL/g) to the vessel and stir for 5 minutes then use to |
| | wash the filter cake. Leave to pull dry for at least 15 minutes |
| 9 | Add polish filtered IPA (0.5 mL/g) to the vessel and stir for 5 minutes then use to |
| | wash the filter cake. Leave to pull dry for at least 30 minutes. |
| 10 | Dry the solid in vacuo at 40° C. until constant weight is achieved. |

¹H NMR (d⁶-DMSO) provided 99.569 wt % Mescaline Hydrochloride and 0.404 wt % isopropanol.

### Example 7—Purication of Mescaline Hydrochloride

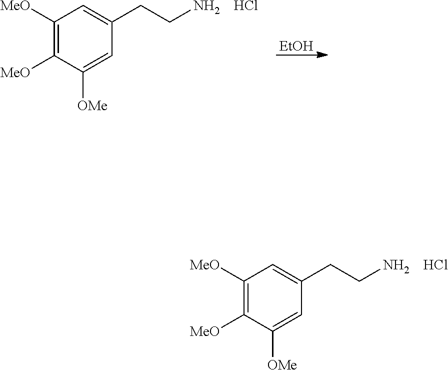 [SMILES: "EtOH" ; Cl ; COc1cc(CCN)cc(OC)c1OC ; Cl ; COc1cc(CCN)cc(OC)c1OC]

Table 4 provides the process for the purification of Mescaline Hydrochloride on a 247 gram scale. Final weight of product was 204 grams (82.5% yield).

TABLE 4
Steps For The Purification of Mescaline Hydrochloride

| Step No. | Process |
|---|---|
| 1 | Add Mescaline HCl salt (1.0 g/g) to the vessel |
| 2 | Polish filter EtOH (5 mL/g) to the vessel and start the stirrer |
| 3 | Heat the suspension to 77-80° C. and stir until a solution is obtained. |
| 4 | Cool the mixture to 0-5° C. over at least 1.5 hours |
| 5 | Stir at 0-5° C. for at least 30 minutes |
| 6 | Filter the mixture |
| 7 | Add polish filtered EtOH (0.5 mL/g) to the vessel and stir for 5 minutes then use |
| | this to wash the filter cake. Allow to pull dry for at least 10 minutes. |
| 8 | Add polish filtered EtOH (0.5 mL/g) to the vessel and stir for 5 minutes then use |
| | this to wash the filter cake. Allow to pull dry for at least 30 minutes. |
| 9 | Dry the solid in vacuo at 40° C. until constant weight is achieved. |
| | A white to off-white solid is obtained. |

¹H NMR (d⁶-DMSO) provided 99.537 wt % Mescaline Hydrochloride and 0.463 wt % ethanol.

A batch analysis provided a final purity of Mescaline hydrochloride of 99.7% by HPLC; Water content by Karl Fischer 0.11 wt %; Residual solvents: 0.08% (836 ppm ethanol); Chloride content by titration: 14.74 wt %; and Elemental Analysis: Li 0.6 ppm and Al<0.1 ppm.

### Example 8—Production Scale Synthesis of Mescaline Hydrochloride

### Step 1: Synthesis of 2-(3,4,5-trimethoxyphenyl)acetamide

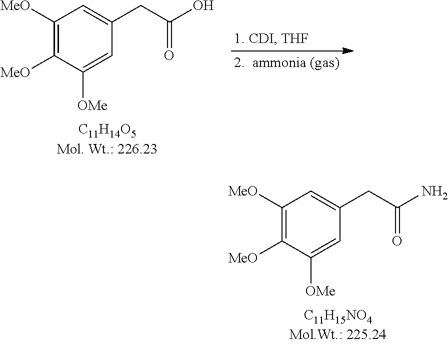 [SMILES: COc1cc(CC(=O)O)cc(OC)c1OC ; COc1cc(CC(N)=O)cc(OC)c1OC]

Four batches of 2-(3,4,5-trimethoxyphenyl)acetamide were synthesized using in a total of 11 kg of the phenyl acetic acid starting material and resulting in 8.655 kg of 2-(3,4,5-trimethoxyphenyl)acetamide (79% active yield). This yield was higher than the anticipated yield of 8256 g (75.4% active yield).

### Batch No. 1 Synthetic Procedure.

To a clean 50 L vessel under nitrogen was charged 3,4,5-trimethoxyphenylacetic acid (2750 g, 12.15 mol) and THF (27.5 L, 10 vol). This was then added with carbonyl diimidazole (CDI, 2365 g, 14.58 mol) portion wise over 40 minutes, maintaining a temperature of between 15 and 25° C. The reaction mixture was then stirred between 15 and 25° C. for 2 hours. HPLC of a sample at this stage was quenched with methanol and indicated adduct formation. The reaction was then sparged with ammonia gas for 50 mins. A sample at this stage was taken and diluted with methanol, the diluted sample was subsequently analyzed via HPLC and indicated that the reaction was complete.

The reaction mixture was then concentrated under vacuum at 40° C. on a rotary evaporator to produce an off white solid. DCM (4 L, 1.45 vol) was charged to the Buchi flask at 40° C. to mostly dissolve the solids. The resulting fine suspension was charged to the 50 L vessel with DCM (23.5 L, 8.54 vol). The reaction mixture was cooled to between 15 and 25° C. and saturated aqueous sodium hydrogen carbonate (13.75 L, 5 vol) was charged, stirred for 30 minutes and the layers were separated. The organic phase was charged back to the vessel and washed twice with saturated sodium hydrogen carbonate (2×8250 mL) and separated. The organic phase was dried over sodium sulphate, filtered, and concentrated under vacuum at 40° C. to give an off white solid. The subsequent wet solid was dried under vacuum in an oven for 16 hours at 40° C. to produce 2-(3,4,5-trimethoxyphenyl)acetamide (2252 g, 82% yield). 1H NMR analysis indicated >95% purity with <2% imidazole remaining. HPLC analysis showed a purity of 98.7%. Karl-fisher (KF) analysis showed <0.3% water present. Advantageously, this product containing imidazole was used in the subsequent production step without the need of additional work-up procedures. Analogous procedures were conducted for batches 2-4 and the results of each batch are summarized below in Table 5.

TABLE 5
Production Overview of Batches 1-4

| | Input | Output | | ¹H NMR | HPLC | |
|---|---|---|---|---|---|---|
| Batch | Mass | mass | Yield | purity | Purity | |
| No. | (g) | (g) | (%) | (%) | (% a/a) | KF |
| 1 | 2750 | 2251 | 82 | >95% | 98.3% | 0.3% |
| 2 | 2750 | 2211 | 80 | >95% | 99.3% | 0.43% |
| 3 | 2750 | 2142 | 78 | >95% | 99% | 0.17% |
| 4 | 2750 | 2051 | 75 | >95% | 98.6% | 0.33% |
| Total | 11000 | 8655 | | | | |

### Step 2: Synthesis of Mescaline

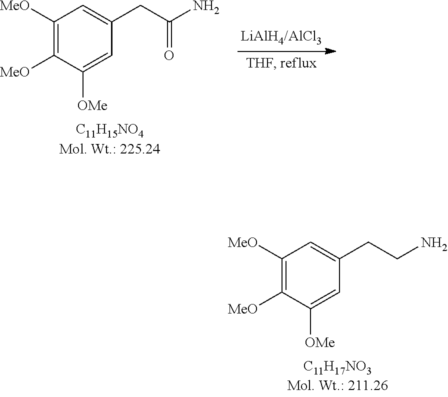 [SMILES: COc1cc(CC(N)=O)cc(OC)c1OC ; COc1cc(CCN)cc(OC)c1OC]

### Work Up Parameters

Initial synthesis of mescaline was conducted at a 530 g scale due to gelation upon a sodium hydroxide quench. Upon scale up, similar gelation was observed indicating that the process was not suitable for 50 L scale up. The material from this batch was processed to afford 338 g of mescaline (Trial batch, 64% against a target of 79.5% yield).

Due to the low yield and a workup that was not suitable for 50 L processing, development of an alternative quench and work up was required. A 50 L batch was performed processing 2000 g of 2-(3,4,5-trimethoxyphenyl)acetamide to produce reasonable amounts of mescaline to attempt various work ups. HPLC analysis of the large-scale batch showed 86.83% product which met the desired completion specification.

From the bulk reaction mixture two 1 L portions were taken through a methanol, ethyl acetate and Rochelle salts work up. Two work procedures were used to evaluate idea parameters for the quench. In work up 1 the reaction mixture was added with the quench solvents, whilst in work up 2 a reverse quench was performed using Batch No. 5.

Work up 1: Afforded 53 g active mescaline (66% active yield). HPLC analysis showed 83% purity with a 5% impurity which was shown to be the acetamide (structure below):

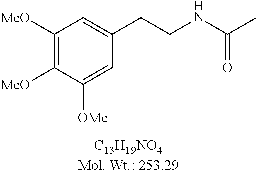 [SMILES: COc1cc(CCNC(C)=O)cc(OC)c1OC]

Work up 2 (reverse quench): Afforded 69 g active mescaline (84% active yield) HPLC analysis showed a 90% purity with none of the acetamide present.

The reverse quench was shown to be better producing a higher yield and purity by HPLC. This procedure was scaled up to 5 L. A total of 348.3 g active of mescaline was isolated. HPLC analysis showed a purity of 90% with none of the acetamide impurity. This procedure was then scaled up to process the remaining 16 L of reaction mixture from Batch No. 6 which afforded 1188 g active mescaline (91.5% active yield based on NMR). HPLC showed a purity of 84.3% with 4.8% of the acetamide impurity previously seen in the 1 L forward quench. Thus, extended processing times on scale up cause the acetamide to form at varying levels. The acetamide is completely purged during the salt formation and purification stages and therefore only causes a modest loss in yield.

### Batch No. 7 Synthetic Procedure

To a clean 20 L vessel under nitrogen was charged THF (5.24 L, 2.62 vol). This was followed by the addition of aluminum trichloride (860 g, 8.58 mol) portion wise over 60 minutes, maintaining temperature <30° C. LiAlH₄ (8.12 L, 2.4M) was then charged dropwise over 80 minutes maintaining a temperature <30° C., a pale grey solution formed. The mixture was cooled to between 15 and 25° C. and stirred for 30 minutes. To a separate 50 L vessel under nitrogen was charged mescaline stage 1 (2000 g, 8.87 mol) and THF (10 L, 5 vol) and the resulting suspension cooled to between 5 and 15ºC. To this cooled suspension the LiAlH₄/AlCl₃ mixture was charged dropwise over 1 hour maintaining temperature <35° C. (exotherm reached 32° C.). The resulting solution was heated to 67° C. and stirred. A sample was taken and analyzed using HPLC which confirmed completion and showed 86.9% product with 0.6% starting material remaining.

The batch was then cooled to room temperature and separated into two portions to perform a split batch work up. For each portion of the reaction mixture (11.5 L) the following procedure was used. The reaction mixture (11.5 L) was charged to a clean 20 L vessel under nitrogen. To the 50 L vessel, methanol (1.3 L, 0.65 vol), ethyl acetate (17.1 L 17.1 vol) and Rochelle salts (aq 53%) (11.5 L, 5.75 vol) were charged and cooled to between 0 and 5° C. The reaction mixture was then charged dropwise via a dip pipe over 2 hours maintaining temperature <30° C. (exotherm reached 26° C.). Once quenched the mixture was stirred for 15 minutes at room temperature and then allowed to separate, ethyl acetate (5.5 L) was charged to aid separation. The resulting aqueous layer was back extracted with ethyl acetate (2×7.36 L). The combined organic phases were dried over sodium sulphate, filtered, and concentrated under vacuum in a rotary evaporator at 50° C. and azeotroped with toluene (3×3 L). This produced 1620 g active mescaline as a pale brown oil (87% active yield based on NMR). HPLC showed a purity of 86.4% with 3% of the acetamide impurity. Batch Nos. 7-8 were similarly processed and the results of each batch are summarized below in Table 6.

TABLE 6
Production Overview of Trial batch and Batches 5-8

| | | | | ¹H NMR | | |
|---|---|---|---|---|---|---|
| | Input | Output | Acetamide | (excluding | HPLC | Yield |
| Batch | (g) | (g) | (% a/a) | solvents) | (% a/a) | (%) |
| Trial | 530 | 338 | 0 | >90% | 89.75% | 68% |
| 5 | 2000 | 1653 | 4.82 | >90% | 84.29% | 88% |
| 6 | 2000 | 1620 | 3.00 | >90% | 86.47% | 87% |
| 7 | 2100 | 1663 | 2.90 | >90% | 82.8% | 85% |
| 8 | 2095 | 1819 | 2.57 | >90% | 86.2% | 93% |
| Total | 8725 | 7093 | | | | |

### Step 3: Synthesis of Mescaline Hydrochloride

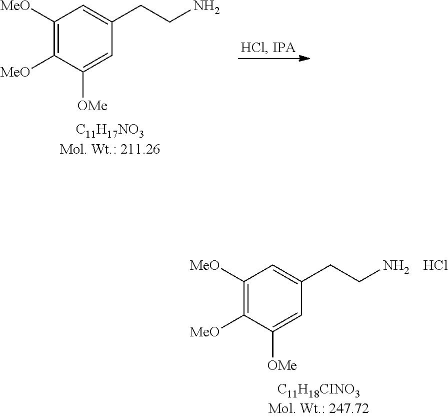

The synthesis of mescaline hydrochloride was split over two batches using 7064 g of mescaline. This resulted in 6390 g mescaline hydrochloride in 76.9% active yield following the procedure previously developed. This stage proceeded as expected and successfully provided more than the target quantity of mescaline hydrochloride (5.07 kg). The amide was reduced to <0.5% in this process.

### Batch No. 10 Synthetic Procedure

Mescaline (5183 g, 24.53 mol) was dissolved in IPA (17.247 L, 3.32 vol) and charged to a clean 50 L under nitrogen, IPA (400 mL, 0.07 vol) was used as a line rinse. The mixture was cooled to between 15-20° C. (17.2° C. actual) and HCl in IPA (4629 mL, 5.3M) was charged dropwise over 60 mins maintaining the temperature <25° C. The reaction mixture was then cooled to 0-5° C. and held for 1 hour, after which it was filtered and washed with IPA (2×2591.5 ml, 2×0.5 vol). The resulting mescaline hydrochloride was oven dried at 40° C. for 2 days to afford 4761 g (78.3% yield). 1H NMR indicated a purity >95% with 0.7% w/w IPA present. HPLC indicated a 94.7% chemical purity with a total of 2.28% of the demethylated species present at RRT 0.72 and RRT 0.77 respectively (structures shown below) and 0.42% of the acetamide at RRT 1.32. A similar procedure was used for Batch No. 9 and the results of each batch are summarized below in Table 7.

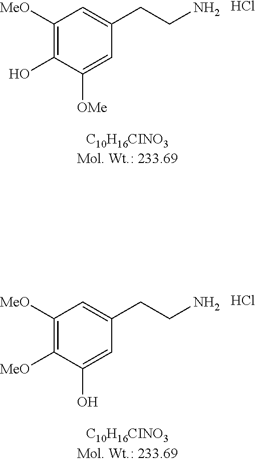 [SMILES: Cl ; COc1cc(CCN)cc(OC)c1O ; Cl ; COc1cc(CCN)cc(O)c1OC]

TABLE 7
Production Overview of Batches 9-10

| | | | | | | Demethylated | | Acetamide |
|---|---|---|---|---|---|---|---|---|
| | | | | Purity by | | Species by | | by HPLC |
| | | | | ¹H NMR | Purity by | HPLC (% a/a) | | (% a/a) |
| | Input | Output | Yield | (excluding | HPLC | RRT | RRT | RRT |
| Batch | (g) | (g) | (%) | solvents) | (%) a/a | 0.72 | 0.77 | 1.31 |
| 9 | 1881 | 1629 | 73.8 | >95% | 94.3% | 0.99 | 0.80 | 0.45% |
| 10 | 5183 | 4761 | 78.3 | >95% | 94.7% | 1.16 | 1.48 | 0.42% |
| Total | 7064 | 6390 | | | | | | |

### Step 4 Purification of Mescaline Hydrochloride

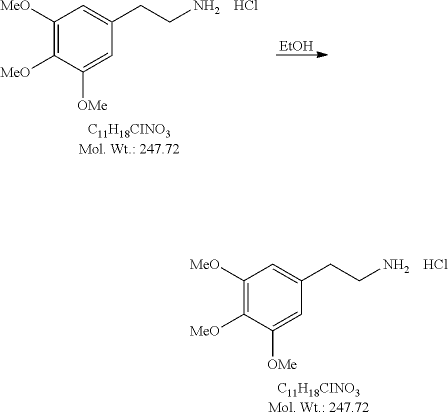 [SMILES: "EtOH" ; Cl ; COc1cc(CCN)cc(OC)c1OC ; Cl ; COc1cc(CCN)cc(OC)c1OC]

The purification of mescaline hydrochloride was split over two batches using 1200 g and 5196 g respectively. This afforded a total of 5324 g mescaline hydrochloride (83.2% active yield) against a target of 4 kg. NMR of both batches indicated a purity of >95% and LC indicated a purity of ≥99%.

### Batch No. 12 Purification Procedure

To a clean 50 L vessel under nitrogen, mescaline. HCl (5196.5 g, 20.97 mol) and ethanol (25.9 L, 5 vol) were charged. The resulting suspension was heated to 77-80° C. until dissolution was achieved (77° C. actual). The reaction was then cooled to 0-5° C. over 2 hours (product precipitates at 65° C.) and held for 30 mins. The batch was then filtered and washed with cooled ethanol (2×2598.2 mL, 2×0.5 vol). The resulting solid was oven dried at 40° C. for 2 days to afford 4360 g (84% yield). A similar procedure was used for Batch No. 11 and the results of each batch are summarized below in Table 8.

TABLE 8
Production Overview of Batches 11-12

| | Input | Output | H NMR (excluding | HPLC | Yield |
|---|---|---|---|---|---|
| Batch | (g) | (g) | solvents) | (% a/a) | (%) |
| 11 | 1200 | 964 | >95% | 99.4% | 80.3 |
| 12 | 5196 | 4360 | >95% | 99.4% | 83.9 |
| Total | 6396 | 5324 | | | |

Batch No. 11 analysis provided a final purity of Mescaline hydrochloride of 99.4% by HPLC; Water content by Karl Fischer 0.3 wt %; Residual solvents: methanol 20 ppm, ethanol 691 ppm, propan-2-ol 3 ppm, THF 7 ppm, toluene 1 ppm; Chloride content by titration: 13.1 wt %; and Elemental Analysis: Li 3.6 ppm and Al 139.5 ppm.

Batch No. 12 analysis provided a final purity of Mescaline hydrochloride of 99.4% by HPLC; Water content by Karl Fischer 0.2 wt %; Residual solvents: methanol 12 ppm, ethanol 689 ppm, propan-2-ol 1 ppm; Chloride content by titration: 14.7 wt %; and Elemental Analysis: Li 0.8 ppm and Al 5.1 ppm.

### Example 9—Safety, Tolerability, Pharmacokinetics, and Pharmacodynamics of Mescaline in Healthy Adults

### Study Design

This trial will follow a double-blind, placebo-controlled, single-ascending dose study in healthy volunteers to assess the safety, tolerability, PK, and PD of orally administered mescaline or a salt thereof.

Mescaline or a salt thereof will be administered in a controlled clinical setting and steps to protect participant safety will include: a preparatory therapy session to prepare participants for possible physiological and psychological effects of mescaline or a salt thereof, continuous in-session monitoring to evaluate physiological effects and provide reassurance and psychological support as needed, and multiple safety follow-up visits post-dosing to assess any ongoing or persisting adverse events, including perceptual disturbances. During the dosing visit, participants will be under direct observation by at least one appropriately trained and qualified study staff member (i.e., dosing session monitor) at all times. Each dose will be administered to a subject following an overnight fast. Dosing session monitors will be licensed healthcare professionals appropriately trained and qualified to monitor participants' psychological and physiological well-being throughout the dosing session.

The trial duration will last up to 52 days for all participants enrolled, which will consist of a screening period, an inpatient treatment period with mescaline or a salt thereof, and a follow-up period. The follow-up visits may be conducted by telephone or as a video call visit in lieu of an in-clinic visit. Participants will report to the clinic the day before dosing and will be domiciled at the clinical research site until post-dosing.

### Safety Monitoring

Safety and tolerability assessments will include monitoring of the following parameters: physical examination, adverse events (AE), 12-lead safety ECGs (e.g., heart rate (HR) (bpm), PR, QRS, QT, QTcF), vital signs (e.g., supine blood pressure, pulse rate, respiratory rate, and body temperature) and clinical laboratory tests (e.g., hematology, biochemistry, and urinalysis). Vital sign and 12-lead safety ECG monitoring will occur pre-dose and post-dose. The subjective state of the subject (e.g., anxiety) will be monitored continuously via AE reporting by subjects and also on fixed post-dose time points with different questionnaires (e.g., BPRS, C-SSRS, STAI).

The pharmacological profile of mescaline can include alterations of perception, consciousness, and the potential for development of acute psychological reactions. Based on the pharmacological profile, dosing session participants will also be continuously monitored for psychological and physiological well-being.

Additionally, prior to release from the clinical site, participants will undergo safety assessments. These assessments can include an evaluation for the presence of any adverse events, treatment-emergent suicidal ideation, or behavior using the C-SSRS. Also, the presence of any psychiatric symptoms will be assessed on using the BPRS (occurrence of psychotic symptoms).

Table 9 provides a summary of the dosing session monitoring parameters.

TABLE 9
Session Monitoring Parameters

| Monitoring | |
|---|---|
| Parameter | Frequency of Collection |
| Psychological | Continuous monitoring for negative psychological |
| well-being | reactions to. The STAI-state subscale will be |
| | administered post-dose. Also, psychological state will |
| | be more extensively assessed using the BPRS, C-SSRS |
| | pre-dose and post-dose. |
| Physiological | Continuous monitoring for physiological adverse. |
| well-being | |
| Vital signs | Pre-dose and post-dose |
| (HR and BP) | |

### Evaluation Endpoints

Safety Endpoints: vital signs (e.g., supine blood pressure, pulse rate, body temperature, respiratory rate); safety laboratory tests (e.g., hematology, biochemistry, and urinalysis); 12-lead safety ECG (e.g., heart rate (HR) (bpm), PR, QRS, QT, QTcF); physical examination; evaluation of the occurrence of treatment-emergent suicidal ideation or behavior using the C-SSRS; occurrence of central 5-HT toxicity (Hunter's criteria and CPK); occurrence of psychotic symptoms (BPRS); or incidence, type, and severity of AEs by system organ class and preferred term.

Psychedelic effects that will not be recorded as AEs: self-limiting changes in affect (including anxiety as a result of the psychedelic “rush”, a sense of loss of control and/or euphoria), perception (including hallucinations and innocuous hallucinatory behavior), cognition (including paranoid ideation), somatic sensations (e.g. abdominal pain or nausea) and experience of the self and/or surroundings that are consistent with mescaline's PK; or persistent positive changes in personality (e.g., increase in openness, extraversion), affect, and cognition that results from the psychedelic experience.

Psychedelic effects that will be recorded as AEs: changes in affect (including fear or anxiety as a result of the psychedelic “rush”, a sense of loss of control and/or euphoria), perception (including hallucinations and innocuous hallucinatory behavior), cognition (including paranoid ideation), somatic sensations (e.g. abdominal pain or nausea) and experience of the self and/or surroundings that persist beyond clearance of mescaline; persistent affective changes such as anxiety as a result of the psychedelic “rush”, a sense of loss of control and/or euphoria; persistent paranoid ideation with potentially dangerous and/or impulsive behavior as a result; potentially dangerous and/or impulsive hallucinatory behavior; potentially dangerous and/or impulsive behavior related to euphoria; emergence of suicidal ideation and/or behavior; or any unexpected subjective psychedelic effects.

Pharmacodynamic Endpoints: Visual Analogue Scales (VAS); Bond and Lader (B&L) for general subjective drug effects or Bowdle for psychedelic drug effects; VAS nausea (subjective nausea); Experience Intensity Questionnaire; Profile of Mood States (POMS); STAI-DY-1 (State anxiety); Psychological Insight Questionnaire (PIQ); Psychological Insight Scale (PIS); Challenging Experience Questionnaire (CEQ); Emotional Breakthrough Inventory (EBI); Persisting Effects Questionnaire (PEQ); Hallucinogenic Rating Scale (HRS); Mystical Experience Questionnaire 30-item (MEQ-30); 5-Dimensional Altered States of Consciousness Rating Scale (5D-ASC); Warwick-Edinburgh Mental Well-Being Scale (WEMWBS); or plasma concentrations of brain-derived neurotrophic factor (BDNF), hypothalamic-pituitary-adrenal (HPA)-axis endocrine measures (e.g., cortisol, ACTH, prolactin), and neuroendocrine measures (e.g., oxytocin).

1. A method for treating a substance use disorder, comprising:
(a) identifying a subject who has a substance use disorder; and
(b) orally administering a pharmaceutical composition comprising synthetic mescaline or
a salt thereof to the subject and accompanying said administration with a therapy session.

2-80. (canceled)

81. The method of claim 1, wherein the synthetic mescaline or a salt thereof is represented by Formula (IV): 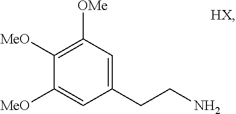 [SMILES: COc1cc(CCN)cc(OC)c1OC ; "HX,"]
wherein HX is an acid and X represents the conjugate base of the acid.

82. The method of claim 1, wherein the pharmaceutical composition comprises an acid addition salt of mescaline selected from an HCl addition salt and a sulfuric acid addition salt.

83. The method of claim 1, wherein the pharmaceutical composition comprises mescaline HCl.

84. The method of claim 1, wherein the synthetic mescaline or a salt thereof is characterized by one or more additional characteristics selected from: (i) between 1 ppm to 50 ppm isopropanol; (ii) between 1 ppm to 50 ppm methanol; (iii) between 500 ppm to 1000 ppm ethanol; (iv) between 0.01 wt % to 0.25 wt % water; (v) between 10 wt % to 20 wt % chloride; (vi) between 0.1 ppm to 10 ppm Li; (vii) between 0.1 ppm to 200 ppm Al; (viii) between 0.1 wt % to 8.0 wt % 2-(3,4,5-trimethoxyphenyl)acetamide; (ix) between 0.01 wt % to 5 wt % 4-(2-aminoethyl)-2,6-dimethoxyphenol; and (x) between 0.01 wt % to 5 wt % 5-(2-aminoethyl)-2,3-dimethoxyphenol.

85. The method of claim 1, wherein the pharmaceutical composition comprises from 50-2000 mg of synthetic mescaline or a salt thereof.

86. The method of claim 85, wherein the pharmaceutical composition comprises from 50-800 mg or from 50-700 mg of synthetic mescaline or a salt thereof.

87. The method of claim 1, wherein the pharmaceutical composition is in a tablet or a capsule.

88. The method of claim 1, wherein the substance use disorder is selected from the group consisting of alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder.

89. The method of claim 88, wherein the substance use disorder is alcohol use disorder.

90. The method of claim 89, wherein the alcohol use disorder is selected from alcohol abuse, alcohol dependence, and alcoholism.

91. The method of claim 1, wherein for step (a), the subject has reduced substance consumption for at least 1, 2, 3, 4, 5, 6, or 7 days, or for 5-12 days, prior to step (b).

92. The method of claim 1, wherein the method further comprises a preparatory therapy session prior to step (b) to prepare the subject for the possible psychological and physiological effects of the administering of step (b).

93. The method of claim 1, wherein step (b) further comprises physiological monitoring of the subject.

94. The method of claim 93, wherein the physiological monitoring comprises monitoring of a physiological parameter selected from supine blood pressure, pulse rate, respiratory rate, and body temperature; or monitoring a plasma biomarker selected from brain-derived neurotrophic factor (BDNF) and hypothalamic-pituitary-adrenal (HPA)-axis endocrine measures.

95. The method of claim 1, wherein step (b) further comprises psychological monitoring of the subject.

96. The method of claim 95, wherein the psychological monitoring comprises monitoring for negative psychological reactions to treatment.

97. The method of claim 1, wherein the method further comprises a mescaline treatment holiday of from 2 weeks to 12 months.

98. A method for treating a substance use disorder, comprising:
(a) administering a first treatment comprising mescaline or a salt thereof and accompanying said first treatment with a first therapy session; and
(b) administering a second treatment 5-12 days after said first treatment, said second treatment comprising mescaline or a salt thereof and accompanying said second treatment with a second therapy session.

99. The method of claim 98, wherein the method further comprises one or more maintenance treatments comprising mescaline or a salt thereof and accompanying said maintenance treatments with a maintenance therapy session.
